(19) United States
(12) Patent Application Publication
(10) Pub. No.: US 2007/0003561 A1
(43) Pub. Date: Jan. 4, 2007

(54) Binding member towards pneumococcus surface adhesin a protein (psaa)

(76) Inventors: Anders Per Sorensen, Hillerod (DK); Thomas Lars Benfield, Copenhagen K (DK); Jens Dilling Lundgren, Charlottenlund (DK); Thomas D. Kempe, Sunnyvale, CA (US)

Correspondence Address: GIFFORD, KRASS, GROH, SPRINKLE & CITKOWSKI, P.C, PO BOX 7021, TROY, MI, 48007-7021, US

(21) Appl. No.: 10/563,976

(22) PCT Filed: Jul. 8, 2004
(86) PCT No.: PCT/DK04/00492

§ 371 Date: Feb. 2, 2006

Related U.S. Application Data
(60) Provisional application No. 60/486,647, filed on Jul. 11, 2003.

(30) Foreign Application Priority Data
Jul. 8, 2003 (DK) PA 2003 01044

(51) Int. Cl. A61K 39/40; C07K 16/12

(52) U.S. Cl. 424/165.1; 530/388.4

(57) ABSTRACT

The present invention relates to a binding member comprising at least one binding domain capable of specifically binding Streptococcus pneumoniae surface adhesion A (PsaA) protein, in particular to a binding member having at least two binding domains, to the use of said binding members in diagnostic methods as well as for treatment. In a preferred embodiment the binding member is an antibody, such as a human antibody, or a fragment thereof, and it may also be a bispecific antibody.

The present invention relates to a binding member comprising at least one binding domain capable of specifically binding Streptococcus pneumoniae surface adhesin A (PsaA) protein, in particular to a binding member having at least two binding domains, to the use of said binding members in diagnostic methods as well as for treatment.

### BACKGROUND

Streptococcus pneumoniae is one of the leading causes of life-threatening bacterial infection. In developing countries it has been estimated that several million children under 5 years of age will die of S. pneumoniae each year (3). In the industrialized world, the incidence of S. pneumoniae pneumonia is 5-10 per 100,000 persons and the case-fatality rate is 5-7%. S. pneumoniae meningitis occurs in 1-2 per 100,000 persons with a case-fatality of 30-40% (14). S. pneumoniae is one of the most frequent causes of bacteremia. S. pneumoniae is the most frequent organism isolated from children with otitis media. App. 75% of all children less than 6 years old will suffer from otitis media.

S. pneumoniae is a gram-positive bacteria that grows in pairs or short chains. The surface is composed of three layers: capsule, cell wall and plasma membrane. The capsule is the thickest layer and completely conceals the inner structures of growing S. pneumoniae. Polymers of repeating units of oligosaccharides (polysaccharides) dominant the capsule. Different serotypes contain ribitol, arabitinol or phosphorylcholine as part of their capsule, resulting in chemical structures that are serotype specific. The cell wall consists of peptidoglycan but also teichoic acid and lipoteichoic acid. The plasma membrane is a double phospholipid membrane that encompasses the cell and anchors various molecules to its surface (2).

At present 90 different types of S. pneumoniae are recognized based on the diversity of the S. pneumoniae capsule (26). The capsule is pivotal in the pathogenesis of S. pneumoniae infections. Antibodies raised against one capsular type offers protection from infection with this type but not against infection with other capsular types. The current 23-valent polysaccharide vaccine offers protection from more than 60-85% of the most frequent serotypes.

The cell wall contains two polysaccarides, C-polysaccharide (C-PS) (teichoic acid and peptidoglycan) and F-antigen (lipoteichoic acid, Forssman antigen) (26). Other bacteria than S. pneumoniae contain C-PS, e.g. alpha-streptococci (12). F-antigen cross-reacts with streptococcal group C polysaccharide (29). Several antibodies to capsular polysaccharides cross-react with C-PS presumably because these are co-valently linked. The protective role of anti-C-PS antibodies is controversial since some studies find them protective in mice (6) and others not (20;28). The lack of protection is believed to be caused by the capsular concealment of C-PS. Antibodies to C-PS may protect hosts infected with acapsular strains or bind to decaying S. pneumoniae that shed their capsule.

Immunization of mice with the F-antigen does not protect against S. pneumoniae infection (4).

Pneumococcal surface adhesin A (PsaA) is a 37-kDa surface protein. A monoclonal antibody towards PsaA reacted with 24 of 24 different encapsulated S. pneumoniae lysates and none of a number of other bacteria (23). RFLP analysis of the psaA gene from 80 strains representing 23 capsule serotypes showed they were highly conserved (24). Immunization of mice with PsaA provided protection against type 3 S. pneumoniae (32). Native PsaA is purified by standard methods (25;33). Recombinant PsaA can be expressed in a baculovirus vector system (10). Anti-rPsaA immune serum conferred protection in mice against S. pneumoniae serotype 6B compared to control mice (10). At least six different monoclonal antibodies have been reported (9;23), and suggested for diagnostic purposes, however treatment of Streptococcus pneumoniae associated diseases have not been suggested with these antibodies.

IgA to PsaA is detectable in saliva from children less than two years (193 of 261) and adults (17 of 17) (34). Anti-PsaA IgG was detectable by EIA in most children less than two years (872 of 1108) and most adults (262/325) (35). Seroconversion was correlated to carrier status, i.e. children who had had with S. pneumoniae cultured from nasopharyngeal or middle ear specimens were more likely to be anti-PsaA IgG positive.

### SUMMARY

The present invention relates to a binding member comprising at least one binding domain capable of specifically binding Streptococcus pneumoniae surface adhesin A (PsaA) protein, wherein the binding member is suitable for use in a pharmaceutical composition for preventing and treating diseases and disorders related to Streptococcus, in particular Streptococcus pneumoniae.

Accordingly, in one embodiment the invention relates to an isolated binding member comprising at least one binding domain capable of specifically binding Streptococcus pneumoniae surface adhesin A (PsaA) protein, said binding domain having a dissociation constant K_{d} for PsaA which is less than 1×10⁻⁶. Preferably the binding member comprising the binding domain has the dissociation constant K_{d} defined above.

Due to the high binding strength the binding member is suitable for use in a pharmaceutical composition.

In another aspect the invention relates to an isolated binding member comprising at least a first binding domain and a second binding domain, said first binding domain being capable of specifically binding Streptococcus pneumoniae surface adhesin A (PsaA) protein.

The binding member according to the invention is preferably an antibody or a fragment of an antibody. The antibody may be produced by any suitable method known to the person skilled in the art, however it is preferred that at least a part of the binding member is produced through a recombinant method. Accordingly, the present invention relates in one aspect to an isolated nucleic acid molecule encoding at least a part of the binding member as defined above, as well as to a vector comprising the nucleic acid molecule defined above, and a host cell comprising the nucleic acid molecule defined above.

The invention further relates to a cell line engineered to express at least a part of the binding member as defined above, and more preferably engineered to express the whole binding member as defined above.

In a further aspect the invention relates to a method of detecting or diagnosing a disease or disorder associated with Pneumococcus in an individual comprising
providing a biological sample from said individual,
adding at least one binding member as defined above to said biological sample
detecting binding members bound to said biological sample, thereby detecting or diagnosing the disease or disorder.

Also, in the method the invention further relates to a kit comprising at least one binding member as defined above, wherein said binding member is labelled, for use in a diagnostic method.

In yet another aspect the invention relates to a pharmaceutical composition comprising at least one binding member as defined above.

Furthermore, the invention relates to the use of a binding member as defined above for the production of a pharmaceutical composition for the treatment or prophylaxis of disorders or diseases associated with Streptococcus pneumoniae, such as pneumonia, meningitis and/or sepsis.

In yet a further aspect the invention relates to a method for treating or preventing an individual suffering from disorders or diseases associated with Streptococcus pneumoniae, such as pneumonia, meningitis and/or sepsis by administering an effective amount of a binding member as defined above.

### DRAWINGS

FIG. 1. Schematic drawing of a Fab fragment.

FIG. 2. Size exclusion HPLC profiles of the F(ab′)₂ fragments of 88.53 (2a) and 5-9A7 (2b).

FIG. 3. Size exclusion HPLC profile of the Fab′ fragment of 88.53.

FIG. 4. Size exclusion HPLC profile of the Fab′ fragment of 5-9A7.

FIG. 5. Size exclusion HPLC profile of 88.53×5-9A7 conjugation mixture.

FIG. 6. Size exclusion HPLC profile of purified 88.53×5-9A7 bispecific antibody.

FIG. 7. Size exclusion HPLC profiles of the F(ab′)₂ fragments of 88.53 (7a) and 14A8 (7b).

FIG. 8. Size exclusion HPLC profile of the Fab′ fragment of 88.53.

FIG. 9. Size exclusion HPLC profile of the Fab′ fragment of 14A8.

FIG. 10. Size exclusion HPLC profile of 88.53×14A8 conjugation mixture.

FIG. 11. Size exclusion HPLC profile of purified 88.53×14A8 bispecific antibody.

FIG. 12. Schematic picture of bispecific antibodies

FIG. 13. Bispecific binding activity of the 88.53×5-9A7 bispecific antibody.

FIG. 14. The 88.53×5-9A7 bispecific antibody binds to CD64 expressed by human CD64 transgenic mice.

FIG. 15. PsaA amino acid sequence having SEQ ID NO: 50.

FIG. 16a. Anti-PsaA 7-1G9 VK, wherein V-segment: L15 and J-segment: JK1

FIG. 16b. Anti-PsaA 7-1G9 VH, wherein V-segment: 4-34, D-segment: unknown, and J-segment: JH4b

FIG. 17a. Anti-PsaA 1-15E5 VK, V-segment: L6, and J-segment: JK4

FIG. 17b. Anti-PsaA 1-15E5 VH, V-segment 3-7, D-segment: 3-10, J-segment: JH6b

FIG. 18a. Anti-PsaA 9A7 VK, V-segment: L6, and J-segment: JK3

FIG. 18b. Anti-PsaA 9A7 VH, V-segment: 3-7, D-segment: 3-10, and J-segment: JH6b

FIG. 19. Four graphs showing the effect of anti-PsaA antibodies on Pneumococcus infection.

FIG. 20. Table for results from in vitro tests.

Sequence listing
Seq ID numbers and names

| | |
|---|---|
| SEQ ID NO 1: | CDR1 Anti-PsaA 7-1G9 VK DNA sequence |
| SEQ ID NO 2: | CDR1 Anti-PsaA 7-1G9 VK amino acid sequence |
| SEQ ID NO 3: | CDR2 Anti-PsaA 7-1G9 VK DNA sequence |
| SEQ ID NO 4: | CDR2 Anti-PsaA 7-1G9 VK amino acid sequence |
| SEQ ID NO 5: | CDR3 Anti-PsaA 7-1G9 VK DNA sequence |
| SEQ ID NO 6: | CDR4 Anti-PsaA 7-1G9 VK amino acid sequence |
| SEQ ID NO 7: | Anti-PsaA 7-1G9 VK DNA sequence |
| SEQ ID NO 8: | Anti-PsaA 7-1G9 VK amino acid sequence |
| SEQ ID NO 9: | CDR1 Anti-PsaA 7-1G9 VH DNA sequence |
| SEQ ID NO 10: | CDR1 Anti-PsaA 7-1G9 VH amino acid sequence |
| SEQ ID NO 11: | CDR2 Anti-PsaA 7-1G9 VH DNA sequence |
| SEQ ID NO 12: | CDR2 Anti-PsaA 7-1G9 VH amino acid sequence |
| SEQ ID NO 13: | CDR1 Anti-PsaA 7-1G9 VH DNA sequence |
| SEQ ID NO 14: | CDR3 Anti-PsaA 7-1G9 VH amino acid sequence |
| SEQ ID NO 15: | Anti-PsaA 7-1G9 VH DNA sequence |
| SEQ ID NO 16: | Anti-PsaA 7-1G9 VH amino acid sequence |
| SEQ ID NO 17: | CDR1 Anti-PsaA 1-15E5 VK DNA sequence |
| SEQ ID NO 18: | CDR1 Anti-PsaA 1-15E5 VK amino acid sequence |
| SEQ ID NO 19: | CDR2 Anti-PsaA 1-15E5 VK DNA sequence |
| SEQ ID NO 20: | CDR2 Anti-PsaA 1-15E5 VK amino acid sequence |
| SEQ ID NO 21: | CDR3 Anti-PsaA 1-15E5 VK DNA sequence |
| SEQ ID NO 22: | CDR3 Anti-PsaA 1-15E5 VK amino acid sequence |
| SEQ ID NO 23: | Anti-PsaA 1-15E5 VK DNA sequence |
| SEQ ID NO 24: | Anti-PsaA 1-15E5 VK amino acid sequence |
| SEQ ID NO 25: | CDR1 Anti-PsaA 1-15E5 VH DNA sequence |
| SEQ ID NO 26: | CDR1 Anti-PsaA 1-15E5 VH amino acid sequence |
| SEQ ID NO 27: | CDR2 Anti-PsaA 1-15E5 VH DNA sequence |
| SEQ ID NO 28: | CDR2 Anti-PsaA 1-15E5 VH amino acid sequence |
| SEQ ID NO 29: | CDR3 Anti-PsaA 1-15E5 VH DNA sequence |
| SEQ ID NO 30: | CDR3 Anti-PsaA 1-15E5 VH amino acid sequence |
| SEQ ID NO 31: | Anti-PsaA 1-15E5 VH DNA sequence |
| SEQ ID NO 32: | Anti-PsaA 1-15E5 VH amino acid sequence |
| SEQ ID NO 33: | CDR1 Anti-PsaA 9A7 VK DNA sequence |
| SEQ ID NO 34: | CDR1 Anti-PsaA 9A7 VK amino acid sequence |
| SEQ ID NO 35: | CDR2 Anti-PsaA 9A7 VK DNA sequence |
| SEQ ID NO 36: | CDR2 Anti-PsaA 9A7 VK amino acid sequence |
| SEQ ID NO 37: | CDR3 Anti-PsaA 9A7 VK DNA sequence |
| SEQ ID NO 38: | CDR3 Anti-PsaA 9A7 VK amino acid sequence |
| SEQ ID NO 39: | Anti-PsaA 9A7 VK DNA sequence |
| SEQ ID NO 40: | Anti-PsaA 9A7 VK amino acid sequence |
| SEQ ID NO 41: | CDR1 Anti-PsaA 9A7 VH DNA sequence |
| SEQ ID NO 42: | CDR1 Anti-PsaA 9A7 VH amino acid sequence |
| SEQ ID NO 43: | CDR2 Anti-PsaA 9A7 VH DNA sequence |
| SEQ ID NO 44: | CDR2 Anti-PsaA 9A7 VH amino acid sequence |
| SEQ ID NO 45: | CDR3 Anti-PsaA 9A7 VH DNA sequence |
| SEQ ID NO 46: | CDR3 Anti-PsaA 9A7 VH amino acid sequence |
| SEQ ID NO 47: | Anti-PsaA 9A7 VH DNA sequence |
| SEQ ID NO 48: | Anti-PsaA 9A7 VH amino acid sequence |
| SEQ ID NO 49: | PsaA A-variant DNA sequence |
| SEQ ID NO 50: | PsaA A-variant amino acid sequence |
| SEQ ID NO 51: | Peptide 9136 |
| SEQ ID NO 52: | Peptide 9137 |
| SEQ ID NO 53: | Peptide 9138 |
| SEQ ID NO 54: | PsaA 1-65 amino acid sequence |
| SEQ ID NO 55: | PsaA DNA sequence |
| SEQ ID NO 56: | PsaA amino acid sequence |

### DETAILED DESCRIPTION OF THE INVENTION

Definitions

Affinity: the strength of binding between receptors and their ligands, for example between an antibody and its antigen.

Avidity: The functional combining strength of an antibody with its antigen which is related to both the affinity of the reaction between the epitopes and paratopes, and the valencies of the antibody and antigen

Amino Acid Residue: An amino acid formed upon chemical digestion (hydrolysis) of a polypeptide at its peptide linkages. The amino acid residues described herein are preferably in the “L” isomeric form. However, residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxy terminus of a polypeptide. In keeping with standard polypeptide, abbreviations for amino acid residues are shown in the following Table of Correspondence:

TABLE OF CORRESPONDENCE

| SYMBOL | | |
|---|---|---|
| 1-Letter | 3-Letter | AMINO ACID |
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | Unknown or other |

It should be noted that all amino acid residue sequences represented herein by formulae have a left-to-right orientation in the conventional direction of amino terminus to carboxy terminus. In addition, the phrase “amino acid residue” is broadly defined to include the amino acids listed in the Table of Correspondence as well as modified and unusual amino acids. Furthermore, it should be noted that a dash- at the beginning or end of an amino acid residue sequence indicates a peptide bond to a further sequence of one or more amino acid residues or a covalent bond to an amino-terminal group such as NH₂ or acetyl or to a carboxy-terminal group such as COOH.

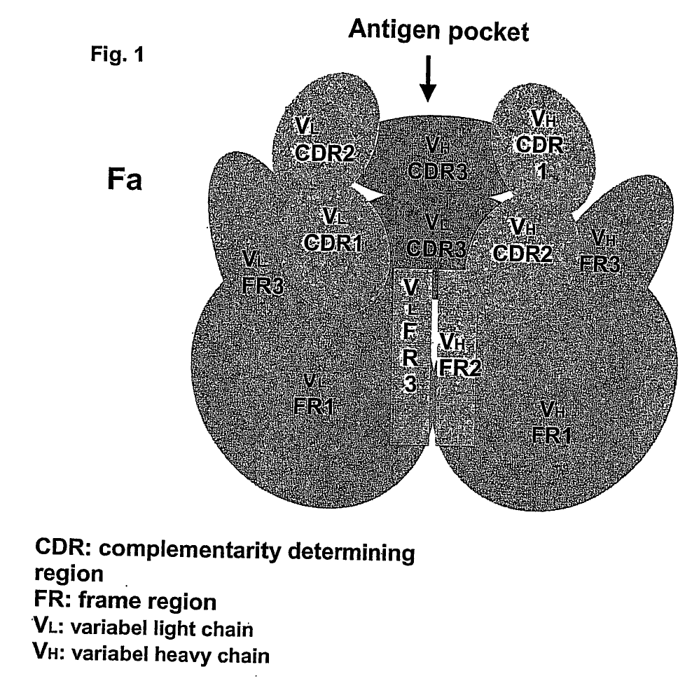

Antibody: The term antibody in its various grammatical forms Is used herein to refer to immunoglobulin molecules and immunologically active portions of immunoglobulin molecules of the compositions of this invention, i.e., molecules that contain an antibody combining site or paratope. Exemplary antibody molecules are intact immunoglobulin molecules, substantially intact immunoglobulin molecules and portions of an immunoglobulin molecule, including those portions known in the art as Fab, Fab′, F(ab′)₂ and Fv. A schematic drawing of Fab is shown in FIG. 1. The term “antibody” as used herein is also intended to include human, single chain and humanized antibodies, as well as binding fragments of such antibodies or modified versions of such antibodies, such as multispecific, bispecific and chimeric molecules having at least one antigen binding determinant derived from an antibody molecule.

Antibody Classes: Depending on the amino acid sequences of the constant domain of their heavy chains, immunoglobulins can be assigned to different classes. There are at least five (5) major classes of immunoglobulins: IgA, IgD, IgE, IgG and IgM, and several of these may be further divided into subclasses (isotypes), e.g. IgG-1, IgG-2, IgG-3 and IgG-4; IgA-1 and IgA-2. The heavy chains constant domains that correspond to the different classes of immunoglobulins are called alpha (α), delta (δ), epsilon (ε), gamma (γ) and mu (μ), respectively. The light chains of antibodies can be assigned to one of two clearly distinct types, called kappa (κ) and lambda (λ), based on the amino sequences of their constant domain. The subunit structures and three-dimensional configurations of different classes of immunoglobulins are well known.

Antibody Combining Site: An antibody combining site is that structural portion of an antibody molecule comprised of a heavy and light chain variable and hypervariable regions that specifically binds (immunoreacts with) an antigen. The term immunoreact in its various forms means specific binding between an antigenic determinant-containing molecule and a molecule containing an antibody combining site such as a whole antibody molecule or a portion thereof. Alternatively, an antibody combining site is known as an antigen binding site.

Base Pair (bp): A partnership of adenine (A) with thymine (T), or of cytosine (C) with guanine (G) in a double stranded DNA molecule. In RNA, uracil (U) is substituted for thymine.

Binding member: a polypeptide that can bind to an epitope on a Streptococcus pneumoniae protein, in particular capable of binding specifically to PsaA.

Binding domain: An antigen binding site which specifically binds an antigen. A binding member may be multispecific and contain two or more binding domains which specifically bind two immunologically distinct antigens.

Chimeric antibody: An antibody in which the variable regions are from one species of animal and the constant regions are from another species of animal. For example, a chimeric antibody can be an antibody having variable regions which derive from a mouse monoclonal antibody and constant regions which are human.

Complementary Bases: Nucleotides that normally pair up when DNA or RNA adopts a double stranded configuration.

Complementarity determining region or CDR: Regions in the V-domains of an antibody that together form the antibody recognizing and binding domain.

Complementary Nucleotide Sequence: A sequence of nucleotides in a single-stranded molecule of DNA or RNA that is sufficiently complementary to that on another single strand to specifically hybridize to it with consequent hydrogen bonding.

Conserved: A nucleotide sequence is conserved with respect to a preselected (reference) sequence if it non-randomly hybridizes to an exact complement of the preselected sequence.

Conservative Substitution: The term conservative substitution as used herein denotes the replacement of an amino acid residue by another, biologically similar residue. Examples of conservative substitutions include the substitution of one hydrophobic residue such as isoleucine, valine, leucine or methionine for another, or the substitution of one polar residue for another, such as the substitution of arginine for lysine, glutamic for aspartic acids, or glutamine for asparagine, and the like. The term conservative substitution also includes the use of a substituted amino acid in place of an unsubstituted parent amino acid provided that molecules having the substituted polypeptide also have the same function.

Constant Region or constant domain or C-domain: Constant regions are those structural portions of an antibody molecule comprising amino acid residue sequences within a given isotype which may contain conservative substitutions therein. Exemplary heavy chain immunoglobulin constant regions are those portions of an immunoglobulin molecule known in the art as CH1, CH2, CH3, CH4 and CH5. An exemplary light chain immunoglobulin constant region is that portion of an immunoglobulin molecule known in the art as C_{L}.

Diabodies: This term refers to a small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad Sci. USA 90: 6444-6448 (1993).

Dissociation constant, Kd: A measure to describe the strength of binding (or affinity or avidity) between receptors and their ligands, for example an antibody and its antigen. The smaller Kd, the stronger binding.

Downstream: Further along a DNA sequence in the direction of sequence transcription or read out, that is travelling in a 3′- to 5′-direction along the non-coding strand of the DNA or 5′- to 3′-direction along the RNA transcript.

Duplex DNA: A double-stranded nucleic acid molecule comprising two strands of substantially complementary polynucleotides held together by one or more hydrogen bonds between each of the complementary bases present in a base pair of the duplex. Because the nucleotides that form a base pair can be either a ribonucleotide base or a deoxyribonucleotide base, the phrase “duplex DNA” refers to either a DNA-DNA duplex comprising two DNA strands (ds DNA), or an RNA-DNA duplex comprising one DNA and one RNA strand.

Fusion Polypeptide: A polypeptide comprised of at least two polypeptides and a linking sequence to operatively link the two polypeptides into one continuous polypeptide. The two polypeptides linked in a fusion polypeptide are typically derived from two independent sources, and therefore a fusion polypeptide comprises two linked polypeptides not normally found linked in nature.

Fv: dual chain antibody fragment containing both a V_{H} and a V_{L}.

Gene: A nucleic acid whose nucleotide sequence codes for an RNA or polypeptide. A gene can be either RNA or DNA.

Human antibody framework: A molecule having an antigen binding site and essentially all remaining immunoglobulin-derived parts of the molecule derived from a human immunoglobulin.

Humanised antibody framework: A molecule having an antigen binding site derived from an immunoglobulin from a non-human species, whereas some or all of the remaining immunoglobulin-derived parts of the molecule is derived from a human immunoglobulin. The antigen binding site may comprise: either a complete variable domain from the non-human immunoglobulin fused onto one or more human constant domains; or one or more of the complementarity determining regions (CDRs) grafted onto appropriate human framework regions in the variable domain. In a humanized antibody, the CDRs can be from a mouse monoclonal antibody and the other regions of the antibody are human.

Hybridization: The pairing of substantially complementary nucleotide sequences (strands of nucleic acid) to form a duplex or heteroduplex by the establishment of hydrogen bonds between complementary base pairs. It is a specific, i.e. non-random, interaction between two complementary polynucleotides that can be competitively inhibited.

Immunoglobulin: The serum antibodies, including IgG, IgM, IgA, IgE and IgD.

Immunoglobulin isotypes: The names given to the Ig which have different H chains, the names are IgG (IgG_{1,2,3,4}), IgM, IgA (IgA_{1,2}), sIgA, IgE, IgD.

Immunologically distinct: The phrase immunologically distinct refers to the ability to distinguish between two polypeptides on the ability of an antibody to specifically bind one of the polypeptides and not specifically bind the other polypeptide.

Individual: A living animal or human in need of susceptible to a condition, in particular an infectious disease” as defined below. The subject is an organism possessing leukocytes capable of responding to antigenic stimulation and growth factor stimulation. In preferred embodiments, the subject is a mammal, including humans and non-human mammals such as dogs, cats, pigs, cows, sheep, goats, horses, rats, and mice. In the most preferred embodiment, the subject is a human.

Infectious disease: a disorder caused by one or more species of Streptococcus, in particular Streptococcus pneumoniae.

Isolated: is used to describe the various binding members, polypeptides and nucleotides disclosed herein, that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified.

Label and indicating means: refer in their various grammatical forms to single atoms and molecules that are either directly or indirectly involved in the production of a detectable signal to indicate the presence of a complex

Monoclonal Antibody: The phrase monoclonal antibody in its various grammatical forms refers to a population of antibody molecules that contains only one species of antibody combining site capable of immunoreacting with a particular antigen. A monoclonal antibody thus typically displays a single binding affinity for any antigen with which it immunoreacts. A monoclonal antibody may contain an antibody molecule having a plurality of antibody combining sites, each immunospecific for a different antigen, e.g., a bispecific monoclonal antibody.

Multimeric: A polypeptide molecule comprising more than one polypeptide. A multimer may be dimeric and contain two polypeptides and a multimer may be trimeric and contain three polypeptides. Multimers may be homomeric and contain two or more identical polypeptides or a multimer may be heteromeric and contain two or more nonidentical polypeptides.

Nucleic Acid: A polymer of nucleotides, either single or double stranded.

Nucleotide: A monomeric unit of DNA or RNA consisting of a sugar moiety (pentose), a phosphate, and a nitrogenous heterocyclic base. The base is linked to the sugar moiety via the glycosidic carbon (1′ carbon of the pentose) and that combination of base and sugar is a nucleoside. When the nucleoside contains a phosphate group bonded to the 3′ or 5′ position of the pentose it is referred to as a nucleotide. A sequence of operatively linked nucleotides is typically referred to herein as a “base sequence” or “nucleotide sequence”, and their grammatical equivalents, and is represented herein by a formula whose left to right orientation is in the conventional direction of 5′-terminus to 3′-terminus.

Nucleotide Analog: A purine or pyrimidine nucleotide that differs structurally from A, T, G, C, or U, but is sufficiently similar to substitute for the normal nucleotide in a nucleic acid molecule.

Pneumococcus: is used synonymously with Streptococcus pneumoniae.

Polyclonal antibody: Polyclonal antibodies Is a mixture of antibody molecules recognising a specific given antigen, hence polyclonal antibodies may recognise different epitopes within said antigen.

Polynucleotide: A polymer of single or double stranded nucleotides. As used herein “polynucleotide” and its grammatical equivalents will include the full range of nucleic acids. A polynucleotide Will typically refer to a nucleic acid molecule comprised of a linear strand of two or more deoxyribonucleotides and/or ribonucleotides. The exact size will depend on many factors, which in turn depends on the ultimate conditions of use, as is well known in the art. The polynucleotides of the present invention, include primers, probes, RNA/DNA segments, oligonucleotides or “oligos” (relatively short polynucleotides), genes, vectors, plasmids, and the like.

Polypeptide: The phrase polypeptide refers to a molecule comprising amino acid residues which do not contain linkages other than amide linkages between adjacent amino acid residues.

Receptor: A receptor is a molecule, such as a protein, glycoprotein and the like, that can specifically (non-randomly) bind to another molecule.

Recombinant DNA (rDNA) molecule: A DNA molecule produced by operatively linking two DNA segments. Thus, a recombinant DNA molecule is a hybrid DNA molecule comprising at least two nucleotide sequences not normally found together in nature. rDNA's not having a common biological origin, i.e., evolutionarily different, are said to be “heterologous”.

Specificity: The term specificity refers to the number of potential antigen binding sites which immunoreact with (specifically bind to) a given antigen in a polypeptide. The polypeptide may be a single polypeptide or may be two or more polypeptides joined by disulfide bonding. A polypeptide may be monospecific and contain one or more antigen binding sites which specifically bind an antigen or a polypeptide may be bispecific and contain two or more antigen binding sites which specifically bind two immunologically distinct antigens. Thus, a polypeptide may contain a plurality of antigen binding sites which specifically bind the same or different antigens.

Serotype: Identification of bacteria within species of Streptococcus, that consist of many strains differing from one another in a variety of characteristics. Commonly used characteristics defining serotypes are particular antigenic molecules.

Single Chain Antibody or scFv: The phrase single chain antibody refers to a single polypeptide comprising one or more antigen binding sites. Furthermore, although the H and L chains of an Fv fragment are encoded by separate genes, they may be linked either directly or via a peptide, for example a synthetic linker can be made that enables them to be made as a single protein chain (known as single chain antibody, sAb; Bird et al. 1988 Science 242:423-426; and Huston et al. 1988 PNAS 85:5879-5883) by recombinant methods. Such single chain antibodies are also encompassed within the term “antibody”, and may be utilized as binding determinants in the design and engineering of a multispecific binding molecule.

Upstream: In the direction opposite to the direction of DNA transcription, and therefore going from 5′ to 3′ on the non-coding strand, or 3′ to 5′ on the mRNA.

Valency: The term valency refers to the number of potential antigen binding sites, i.e. binding domains, in a polypeptide. A polypeptide may be monovalent and contain one antigen binding site or a polypeptide may be bivalent and contain two antigen binding sites. Additionally, a polypeptide may be tetravalent and contain four antigen binding sites. Each antigen binding site specifically binds one antigen. When a, polypeptide comprises more than one antigen binding site, each antigen binding site may specifically bind the same or different antigens. Thus, a polypeptide may contain a plurality of antigen binding sites and therefore be multivalent and a polypeptide may specifically bind the same or different antigens.

V-domain: Variable domain are those structural portions of an antibody molecule comprising amino acid residue sequences forming the antigen binding sites. An exemplary light chain immunoglobulin variable region is that portion of an immunoglobulin molecule known in the art as V_{L}.

V_{L}: Variable domain of the light chain

V_{H}: Variable domain of the heavy chain

Vector: A rDNA molecule capable of autonomous replication in a cell and to which a DNA segment, e.g., gene or polynucleotide, can be operatively linked so as to bring about replication of the attached segment. Vectors capable of directing the expression of genes encoding for one or more polypeptides are referred to herein as “expression vectors”. Particularly important vectors allow cloning of cDNA (complementary DNA) from mRNAs produced using reverse transcriptase.

Description

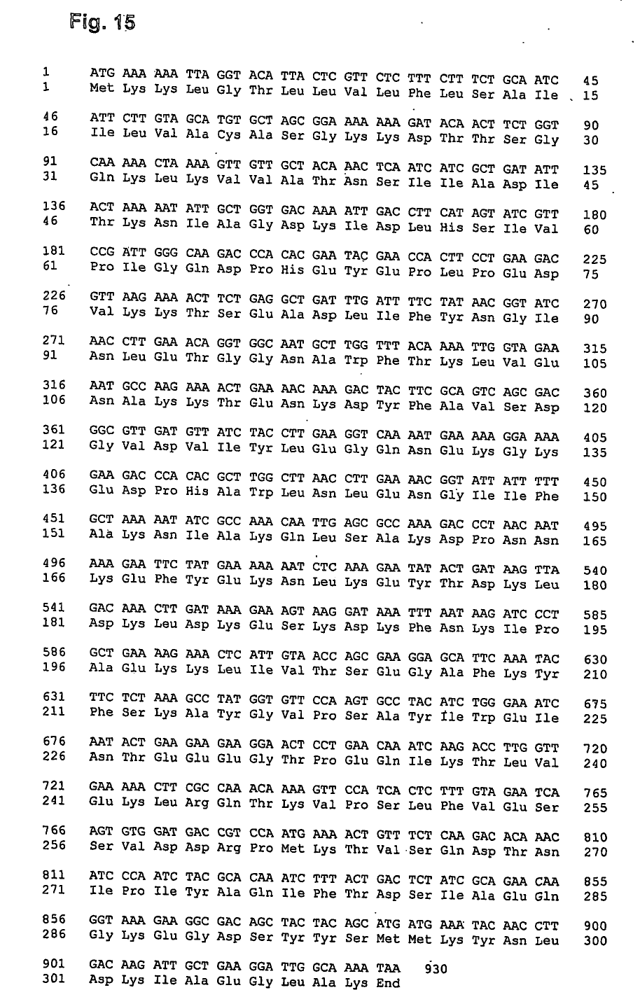

As described above, the present invention relates to binding members, in particular antibodies or fragments thereof capable of specifically recognising and binding to a Streptococcus pneumoniae protein, more specifically to Pneumococcus surface adhesin A protein, PsaA. The binding members according to the invention are particularly useful in the treatment of diseases caused by Streptococcus pneumoniae, as well as for being employed in diagnostic methods and kits for detecting the bacteria. The Penumococcus surface adhesin A protein is preferably a polypeptide having the amino acid sequence shown FIG. 15 (SEQ ID NO: 50) or SEQ ID NO: 56.

Thus, the binding member according to the invention should preferably be immunologically active, for example as an antibody, such as being capable of binding to an antigen and presenting the antigen to immunoactive cells, thereby facilitating phagocytosis of said antigen.

In particular the binding member is an antibody, such as any suitable antibody known in the art, in partucular antibodies as defined herein, such as antibodies or immunologically active fragments of antibodies, or single chain antibodies. Antibody molecules are typically Y-shaped molecules whose basic unit consist of four polypeptides, two identical heavy chains and two identical light chains, which are covalently linked together by disulfide bonds. Each of these chains is folded In discrete domains. The C-terminal regions of both heavy and light chains are conserved in sequence and are called the constant regions, also known as C-domains. The N-terminal regions, also known as V-domains, are variable in sequence and are responsible for the antibody specificity. The antibody specifically recognizes and binds to an antigen mainly through six short complementarity determining regions located in their V-domains (see FIG. 1).

The antibodies according to the invention are especially useful, since they have a strong affinity towards the PsaA.

Accordingly, the binding members according to the invention have a binding domain having a dissociation constant K_{d} for PsaA which is less than 1×10⁻⁸M. More preferably the dissociation constant K_{d} for PsaA which is less than 1×10⁻⁷M, more preferably less than 1×10⁻⁸M, more preferably less than 5×10⁻⁸M, more preferably less than 1×10⁻⁹M, more preferably less than 5×10⁻⁹M more preferably less than 1×10⁻¹⁰M.

The affinity of the binding member towards the PsaA is preferably measured as described in Example 3.

The binding member is preferably an isolated binding member as defined above, and more preferably an isolated, pure binding member.

Complementarity-Determining Regions

Without being bound by theory it is believed that the high binding strength is caused by incorporating into the binding domain an amino acid sequence having one or more of the following motifs of the sequences shown below.

More specifically the binding domain preferably comprises a CDR1 region comprising a sequence selected from

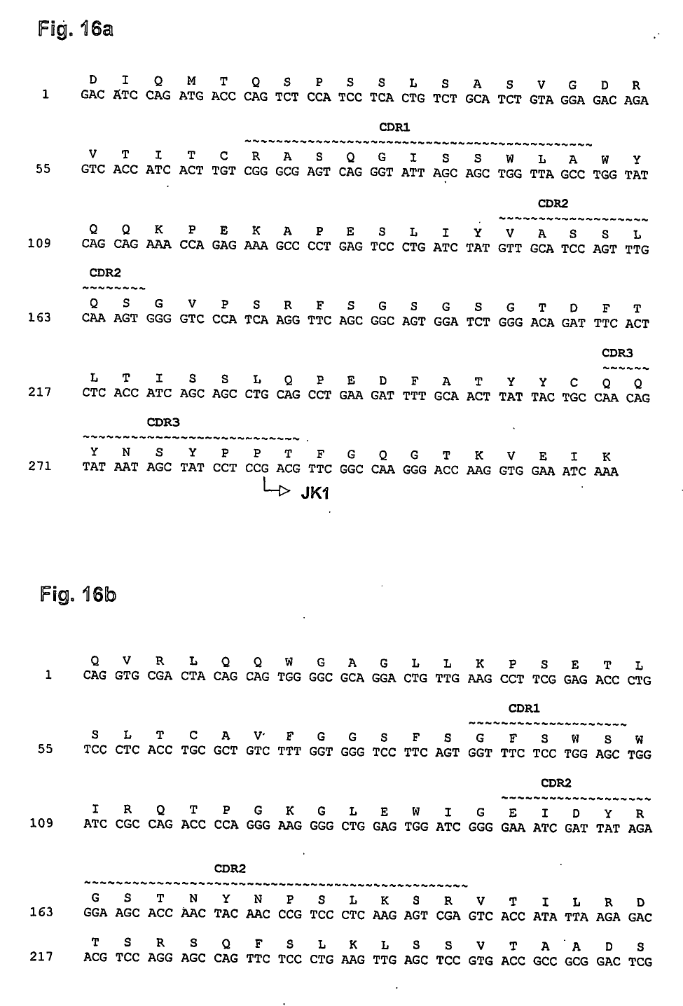

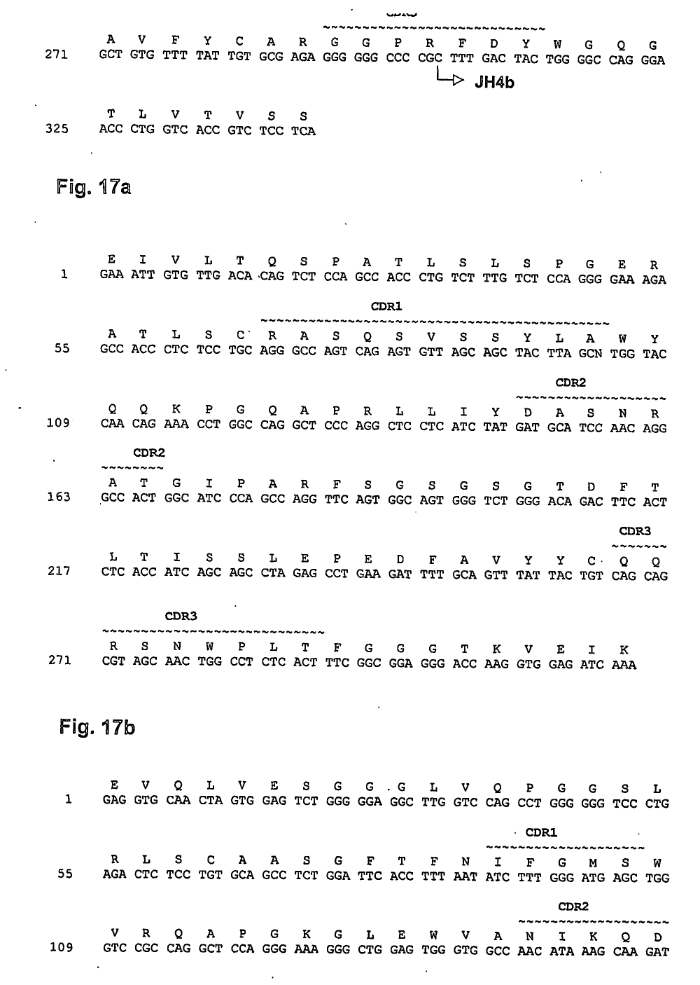

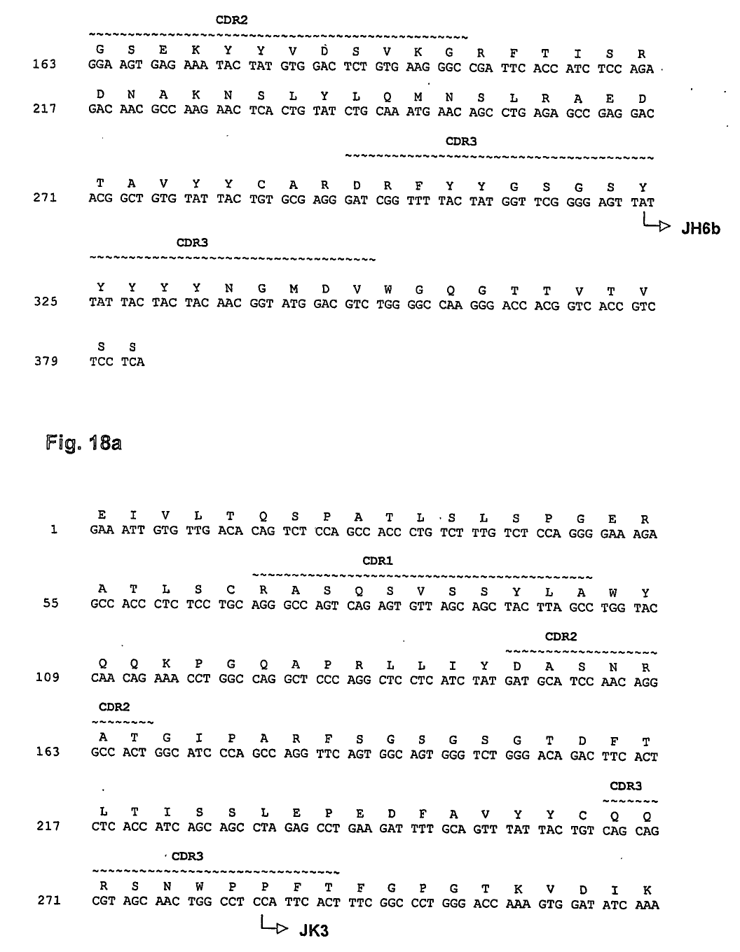

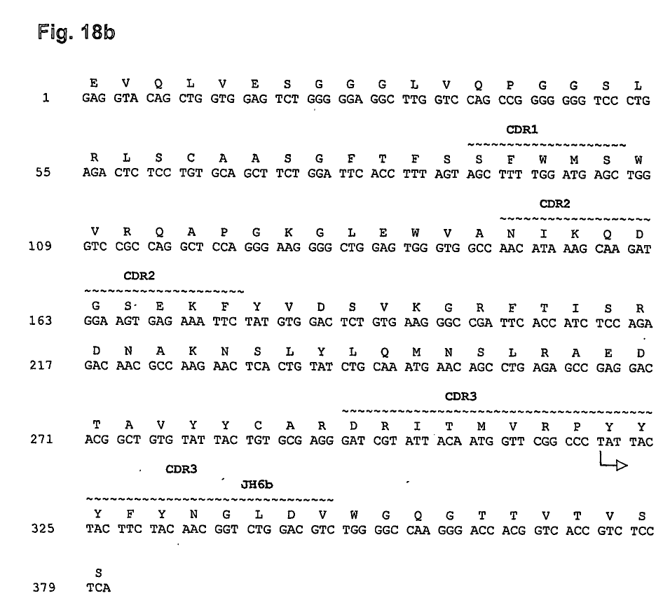

SEQ ID NO 2: CDR1 OF AMINO ACID SEQUENCE IN FIG. 16A

SEQ ID NO 10: CDR1 OF AMINO ACID SEQUENCE IN FIG. 16B

SEQ ID NO 18: CDR1 OF AMINO ACID SEQUENCE IN FIG. 17A

SEQ ID NO 26: CDR1 OF AMINO ACID SEQUENCE IN FIG. 17B

SEQ ID NO 34: CDR1 OF AMINO ACID SEQUENCE IN FIG. 18A

SEQ ID NO 42: CDR1 OF AMINO ACID SEQUENCE IN FIG. 18A

And/or the binding domain preferably comprises a CDR2 region comprising a sequence selected from

SEQ ID NO 4: CDR2 OF AMINO ACID SEQUENCE IN FIG. 16A

SEQ ID NO 12: CDR2 OF AMINO ACID SEQUENCE IN FIG. 16B

SEQ ID NO 20: CDR2 OF AMINO ACID SEQUENCE IN FIG. 17A

SEQ ID NO 28: CDR2 OF AMINO ACID SEQUENCE IN FIG. 17B

SEQ ID NO 36: CDR2 OF AMINO ACID SEQUENCE IN FIG. 18A

SEQ ID NO 44: CDR2 OF AMINO ACID SEQUENCE IN FIG. 18B

And/or the binding domain preferably comprises a CDR3 region comprising a sequence selected from

SEQ ID NO 6: CDR3 OF AMINO ACID SEQUENCE IN FIG. 16A

SEQ ID NO 14: CDR3 OF AMINO ACID SEQUENCE IN FIG. 16B

SEQ ID NO 22: CDR3 OF AMINO ACID SEQUENCE IN FIG. 17A

SEQ ID NO 30: CDR3 OF AMINO ACID SEQUENCE IN FIG. 17B

SEQ ID NO 38: CDR3 OF AMINO ACID SEQUENCE IN FIG. 18A

SEQ ID NO 46: CDR3 OF AMINO ACID SEQUENCE IN FIG. 18B

More preferably the variable part of the binding domain comprises a sequence selected from

SEQ ID NO 8: AMINO ACID SEQUENCE IN FIG. 16A

SEQ ID NO 16: AMINO ACID SEQUENCE IN FIG. 16B

SEQ ID NO 24: AMINO ACID SEQUENCE IN FIG. 17A

SEQ ID NO 32: AMINO ACID SEQUENCE IN FIG. 17B

SEQ ID NO 40: AMINO ACID SEQUENCE IN FIG. 18A

SEQ ID NO 48: AMINO ACID SEQUENCE IN FIG. 18B

or a homologue thereof, wherein a homologue is as defined elsewhere herein.

More preferably the binding domain comprises at least one of the amino acid sequence sets selected from the group of

the amino acid sequence sets SEQ ID NO 2 or a homologue thereof, SEQ ID NO 4 or a homologue thereof, and SEQ ID NO 6 or a homologue thereof, or the amino acid sequence sets SEQ ID NO 10 or a homologue thereof, SEQ ID NO 12 or a homologue thereof, and SEQ ID NO 14 or a homologue thereof, or the amino acid sequence sets SEQ ID NO 18 or a homologue thereof, SEQ ID NO 20 or a homologue thereof, and SEQ ID NO 22 or a homologue thereof, or the amino acid sequence sets SEQ ID NO 26 or a homologue thereof, SEQ ID NO 28 or a homologue thereof, and SEQ ID NO 30 or a homologue thereof, or the amino acid sequence sets SEQ ID NO 34 or a homologue thereof, SEQ ID NO 36 or a homologue thereof, and SEQ ID NO 38 or a homologue thereof, or the amino acid sequence sets SEQ ID NO 42 or a homologue thereof, SEQ ID NO 44 or a homologue thereof, and SEQ ID NO 46 or a homologue thereof.

In the amino acid sequence sets above, the amino acid sequences are preferably arranged in the binding domain as CDR1, CDR2 and CDR3, i.e. spaced apart by other amino acid sequences.

The homology of any one of the homologues described above preferably confers the binding domain comprising one or more homologues with a dissociation constant K_{d} for PsaA as defined above.

Identity and Homology

The term “identity” or “homology” shall be construed to mean the percentage of amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Neither N— or C-terminal extensions nor insertions shall be construed as reducing identity or homology. Methods and computer programs for the alignment are well known in the art. Sequence identity may be measured using sequence analysis software (e.g., Sequence Analysis Software Package, Genetics Computer Group, University of Wisconsin Biotechnology Center, 1710 University Ave., Madison, Wis. 53705). This software matches similar sequences by assigning degrees of homology to various substitutions, deletions, and other modifications.

A homologue of one or more of the sequences specified herein may vary in one or more amino acids as compared to the sequences defined, but is capable of performing the same function, i.e. a homologue may be envisaged as a functional equivalent of a predetermined sequence.

As described above a homologue of any of the predetermined sequences herein may be defined as:
i) homologues comprising an amino acid sequence capable of recognising an antigen also being recognised by the predetermined amino acid sequence, and/or
ii) homologues comprising an amino acid sequence capable of binding selectively to an antigen, wherein said antigen is also bound selectively by a predetermined sequence, and/or
iii) homologues having a substantially similar or higher binding affinity to PsaA as a binding domain comprising a predetermined sequence, such as SEQ ID NO 8.

Examples of homologues comprises one or more conservative amino acid substitutions including one or more conservative amino acid substitutions within the same group of predetermined amino acids, or a plurality of conservative amino acid substitutions, wherein each conservative substitution is generated by substitution within a different group of predetermined amino acids.

Homologues may thus comprise conservative substitutions independently of one another, wherein at least one glycine (Gly) of said homologue is substituted with an amino acid selected from the group of amino acids consisting of Ala, Val, Leu, and Ile, and independently thereof, homologues, wherein at least one of said alanines (Ala) of said homologue thereof is substituted with an amino acid selected from the group of amino acids consisting of Gly, Val, Leu, and lie, and independently thereof, homologues, wherein at least one valine (Val) of said homologue thereof is substituted with an amino acid selected from the group of amino acids consisting of Gly, Ala, Leu, and Ile, and independently thereof, homologues thereof, wherein at least one of said leucines (Leu) of said homologue thereof is substituted with an amino acid selected from the group of amino acids consisting of Gly, Ala, Val, and Ile, and Independently thereof, homologues thereof, wherein at least one isoleucine (Ile) of said homologues thereof is substituted with an amino acid selected from the group of amino acids consisting of Gly, Ala, Val and Leu, and independently thereof, homologues thereof wherein at least one of said aspartic acids (Asp) of said homologue thereof is substituted with an amino acid selected from the group of amino acids consisting of Glu, Asn, and Gin, and independently thereof, homologues thereof, wherein at least one of said phenylalanines (Phe) of said homologues thereof is substituted with an amino acid selected from the group of amino acids consisting of Tyr, Trp, His, Pro, and preferably selected from the group of amino acids consisting of Tyr and Trp, and independently thereof, homologues thereof, wherein at least one of said tyrosines (Tyr) of said homologues thereof is substituted with an amino acid selected from the group of amino acids consisting of Phe, Trp, His, Pro, preferably an amino acid selected from the group of amino acids consisting of Phe and Trp, and independently thereof, homologues thereof, wherein at least one of said arginines (Arg) of said fragment is substituted with an amino acid selected from the group of amino acids consisting of Lys and His, and independently thereof, homologues thereof, wherein at least one lysine (Lys) of said homologues thereof is substituted with an amino acid selected from the group of amino acids consisting of Arg and His, and independently thereof, homologues thereof, wherein at least one of said aspargines (Asn) of said homologues thereof is substituted with an amino acid selected from the group of amino acids consisting of Asp, Glu, and Gln, and independently thereof, homologues thereof, wherein at least one glutamine (Gln) of said homologues thereof is substituted with an amino acid selected from the group of amino acids consisting of Asp, Glu, and Asn, and independently thereof, homologues thereof, wherein at least one proline (Pro) of said homologues thereof is substituted with an amino acid selected from the group of amino acids consisting of Phe, Tyr, Trp, and His, and independently thereof, homologues thereof, wherein at least one of said cysteines (Cys) of said homologues thereof is substituted with an amino acid selected from the group of amino acids consisting of Asp, Glu, Lys, Arg, His, Asn, Gln, Ser, Thr, and Tyr.

Conservative substitutions may be Introduced in any position of a preferred predetermined sequence. It may however also be desirable to introduce non-conservative substitutions, particularly, but not limited to, a non-conservative substitution in any one or more positions.

A non-conservative substitution leading to the formation of a functionally equivalent homologue of the sequences herein would for example i) differ substantially in polarity, for example a residue with a non-polar side chain (Ala, Leu, Pro, Trp, Val, Ile, Leu, Phe or Met) substituted for a residue with a polar side chain such as Gly, Ser, Thr, Cys, Tyr, Asn, or Gin or a charged amino acid such as Asp, Glu, Arg, or Lys, or substituting a charged or a polar residue for a non-polar one; and/or ii) differ substantially in its effect on polypeptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

Substitution of amino acids may in one embodiment be made based upon their hydrophobicity and hydrophilicity values and the relative similarity of the amino acid side-chain substituents, including charge, size, and the like. Exemplary amino acid substitutions which take various of the foregoing characteristics into consideration are well known to those of skill in the art and include: arginine and lysine; glutamate and aspartate; serine and threonine; glutamine and asparagine; and valine, leucine and isoleucine.

In a preferred embodiment the binding domain comprises a homologue having an amino acid sequence at least 60% homologous to a sequence selected from SEQ ID NO 2, SEQ ID NO 4, SEQ ID NO 6 and SEQ ID NO 8.

More preferably the homology is at least 65%, such as at least 70% homologous, such as at least 75% homologous, such as at least 80% homologous, such as at least 85% homologous, such as at least 90% homologous, such as at least 95% homologous, such as at least 98% homologous to a sequence selected from SEQ ID NO 2, SEQ ID NO 4, SEQ ID NO 6 and SEQ ID NO 8.

In a more preferred embodiment the percentages mentioned above relates to the Identity of the sequence of a homologue as compared to a sequence selected from SEQ ID NO 2. SEQ ID NO 4, SEQ ID NO 6 and SEQ ID NO 8.

Epitopes

The antibodies according to the present Invention preferably recognize and bind to an epitope localised in the N-terminal part of PsaA corresponding to N-terminal amino acid residues 1-150 of SEQ ID NO 50 or SEQ ID NO: 56. Preferably, the epitope is localised in the N-terminal part of PsaA corresponding to N-terminal amino acid residues 1-100 of SEQ ID NO 50 or SEQ ID NO: 56, more preferably in the N-terminal part of PsaA corresponding to N-terminal amino acid residues 1-65 of SEQ ID NO 50 or SEQ ID NO: 56.

In a preferred embodiment, the antibody recognizes and binds to a fragment selected from the group of fragments having a sequence corresponding to SEQ ID NO 51, SEQ ID NO 52 or SEQ ID NO 53.

Furthermore, it is preferred that the antibody according to the invention recognizes and binds to an epitope wherein said epitope is also recognized by an antibody having a variable part comprising a sequence selected from the group of

SEQ ID NO 8: AMINO ACID SEQUENCE IN FIG. 16A

SEQ ID NO 16: AMINO ACID SEQUENCE IN FIG. 16B

SEQ ID NO 24: AMINO ACID SEQUENCE IN FIG. 17A

SEQ ID NO 32: AMINO ACID SEQUENCE IN FIG. 17B

SEQ ID NO 40: AMINO ACID SEQUENCE IN FIG. 18A

SEQ ID NO 48: AMINO ACID SEQUENCE IN FIG. 18B

Serotypes

As described above, 90 different serotypes of Streptococcus pneumoniae have been identified. It is preferred that the binding member according to this invention is capable of binding PsaA from two or more different Pneumococcus serotypes, such as from three or more different Pneumococcus serotypes, such as from four or more different Pneumococcus serotypes, such as from five or more different Pneumococcus serotypes. Most preferably the binding member according to the invention is capable of recognising and binding Pneumococcus from essentially all serotypes.

Monoclonal/Polyclonal Antibodies

In one embodiment of the invention, the binding member is an antibody, wherein the antibody may be a polyclonal or a monoclonal antibody derived from a mammal or mixtures of monoclonal antibodies. In a preferred embodiment the binding member is a monoclonal antibody or a fragment thereof. The antibody may be any kind of antibody, however it is preferably a IgG antibody. More preferably the antibody is a IgG1 antibody or a fragment thereof.

Monoclonal antibodies (Mab's) are antibodies, wherein every antibody molecule Is similar and thus recognises the same epitope. Monoclonal antibodies are in general produced by a hybridoma cell line. Methods of making monoclonal antibodies and antibody-synthesizing hybridoma cells are well known to those skilled in the art. Antibody-producing hybridomas may for example be prepared by fusion of an antibody-producing B lymphocyte with an immortalized cell line.

A monoclonal antibody can be produced by the following steps. In all procedures, an animal is immunized with an antigen such as a protein (or peptide thereof as described above for preparation of a polyclonal antibody. The immunization is typically accomplished by administering the immunogen to an immunologically competent mammal in an immunologically effective amount, i.e., an amount sufficient to produce an immune response. Preferably, the mammal is a rodent such as a rabbit, rat or mouse. The mammal is then maintained on a booster schedule for a time period sufficient for the mammal to generate high affinity antibody molecules as described. A suspension of antibody-producing cells is removed from each immunized mammal secreting the desired antibody. After a sufficient time to generate high affinity antibodies, the animal (e.g., mouse) is sacrificed and antibody-producing lymphocytes are obtained from one or more of the lymph nodes, spleens and peripheral blood. Spleen cells are preferred, and can be mechanically separated into individual cells in a physiological medium using methods well known to one of skill in the art. The antibody-producing cells are immortalized by fusion to cells of a mouse myeloma line. Mouse lymphocytes give a high percentage of stable fusions with mouse homologous myelomas, however rat, rabbit and frog somatic cells can also be used. Spleen cells of the desired antibody-producing animals are immortalized by fusing with myeloma cells, generally in the presence of a fusing agent such as polyethylene glycol. Any of a number of myeloma cell lines suitable as a fusion partner are used with to standard techniques, for example, the P3-NS1/1-Ag4-1, P3-x63-Ag8.653 or Sp2/O-Ag14 myeloma lines, available from the American Type Culture Collection (ATCC), Rockville, Md.

Monoclonal antibodies can also be generated by other methods well known to those skilled in the art of recombinant DNA technology. An alternative method, referred to as the “combinatorial antibody display” method, has been developed to identify and isolate antibody fragments having a particular antigen specificity, and can be utilized to produce monoclonal antibodies.

Polyclonal antibodies is a mixture of antibody molecules recognising a specific given antigen, hence polyclonal antibodies may recognise different epitopes within said antigen. In general polyclonal antibodies are purified from serum of a mammal, which previously has been immunized with the antigen. Polyclonal antibodies may for example be prepared by any of the methods described in Antibodies: A Laboratory Manual, By Ed Harlow and David Lane, Cold Spring Harbor Laboratory Press, 1988. Polyclonal antibodies may be derived from any suitable mammalian species, for example from mice, rats, rabbits, donkeys, goats, and sheep.

Specificity

The binding member may be monospecific towards the PsaA protein, wherein specificity towards the PsaA protein means that the binding member immunoreacts with PsaA protein. In another embodiment the binding member is bispecific or multispecific having at least one portion being specific towards the PsaA protein.

Monovalent Antibodies

The monospecific binding member may be monovalent, i.e. having only one binding domain.

For a monovalent antibody, the immunoglobulin constant domain amino acid residue sequences comprise the structural portions of an antibody molecule known in the art as CH1, CH2, CH3 and CH4. Preferred are those binding members which are known in the art as C_{L}. Preferrred C_{L} polypeptides are selected from the group consisting of Cₖₐₚₚₐ. and C_{lambda}.

Furthermore, insofar as the constant domain can be either a heavy or light chain constant domain (C_{H} or C_{L}, respectively), a variety of monovalent binding member compositions are contemplated by the present invention. For example, light chain constant domains are capable of disulfide bridging to either another light chain constant domain, or to a heavy chain constant domain. In contrast, a heavy chain constant domain can form two independent disulfide bridges, allowing for the possibility of bridging to both another heavy chain and to a light chain, or to form polymers of heavy chains.

Thus, in another embodiment, the Invention contemplates a composition comprising a monovalent polypeptide wherein the constant chain domain C has a cysteine residue capable of forming at least one disulfide bridge, and where the composition comprises at least two monovalent polypeptides covalently linked by said disulfide bridge.

In preferred embodiments, the constant chain domain C can be either C_{L} or C_{H}. Where C is C_{L}, the C_{L} polypeptide is preferably selected from the group consisting of Cₖₐₚₚₐ and C_{lambda}.

In another embodiment, the invention contemplates a binding member composition comprising a monovalent polypeptide as above except where C is C_{L} having a cysteine residue capable of forming a disulfide bridge, such that the composition contains two monovalent polypeptides covalently linked by said disulfide bridge.

Multivalent

In another embodiment of the invention the binding member is a multivalent binding member having at least two binding domains. The binding domains may have specificity for the same ligand or for different ligands.

Multispecificity, Including Bispecificity

In a preferred embodiment the Instant Invention relates to multispecific binding members, which have affinity for and are capable of binding at least two different entities. Multispecific binding members can include bispecific binding members.

In one embodiment the multispecific molecule- is a bispecific antibody (BsAb), which carries at least two different binding domains, at least one of which is of antibody origin.

A bispecific molecule of the invention can also be a single chain bispecific molecule, such as a single chain bispecific antibody, a single chain bispecific molecule comprising one single chain antibody and a binding domain, or a single chain bispecific molecule comprising two binding domains. Multispecific molecules can also be single chain molecules or may comprise at least two single chain molecules.

The multispecific, including bispecific antibodies may be produced by any suitable manner known to the person skilled in the art.

The traditional approach to generate bispecific whole antibodies was to fuse two hybridoma cell lines each producing an antibody having the desired specificity. Because of the random association of immunoglobulin heavy and light chains, these hybrid hybridomas produce a mixture of up to 10 different heavy and light chain combinations, only one of which is the bispecific antibody. Therefore, these bispecific antibodies have to be purified with cumbersome procedures, which considerably decrease the yield of the desired product.

Alternative approaches include In-vitro linking of two antigen specificities by chemical cross-linking of cysteine residues either in the hinge or via a genetically introduced C-terminal Cys as described above. An improvement of such in vitro assembly was achieved by using recombinant fusions of Fab's with peptides that promote formation of heterodimers. However, the yield of bispecific product in these methods is far less than 100%.

A more efficient approach to produce bivalent or bispecific antibody fragments, not involving In vitro chemical assembly steps, was described by Holliger et al. (1993). This approach takes advantage of the observation that scFv's secreted from bacteria are often present as both monomers and dimers. This observation suggested that the V_{H} and VL of different chains can pair, thus forming dimers and larger complexes. The dimeric antibody fragments, also named “diabodies” by Hollinger et al., in fact are small bivalent antibody fragments that assembled in vivo. By linking the V_{H} and VL of two different antibodies 1 and 2, to form “cross-over” chains V_{H} 1 VL 2 and V_{H} 2-VL 1, the dimerisation process was shown to reassemble both antigen-binding sites. The affinity of the two binding sites was shown to be equal to the starting scFv's, or even to be 10-fold increased when the polypeptide linker covalently linking V_{H} and VL was removed, thus generating two proteins each consisting of a V_{H} directly and covalently linked to a VL not pairing with the V_{H}. This strategy of producing bispecific antibody fragments was also described in several patent applications. Patent application WO 94/09131 (SCOTGEN LTD; priority date Oct. 15, 1992) relates to a bispecific binding protein in which the binding domains are derived from both a V_{H} and a VL region either present at two chains or linked in an scFv, whereas other fused antibody domains, e.g. C-terminal constant domains, are used to stabilise the dimeric constructs. Patent application WO 94/13804 (CAMBRIDGE ANTIBODY TECHNOLOGY/MEDICAL RESEARCH COUNCIL; first priority date Dec. 4, 1992) relates to a polypeptide containing a V_{H} and a VL which are incapable of associating with each other, whereby the V-domains can be connected with or without a linker.

Mallender and Voss, 1994 (also described in patent application WO 94/13806; DOW CHEMICAL CO; priority date Dec. 11, 1992) reported the in vivo production of a single-chain bispecific antibody fragment in E. coli. The bispecificity of the bivalent protein was based on two previously produced monovalent scFv molecules possessing distinct specificities, being linked together at the genetic level by a flexible polypeptide linker. Traditionally, whenever single-chain antibody fragments are referred to, a single molecule consisting of one heavy chain linked to one (corresponding) light chain in the presence or absence of a polypeptide linker is implicated. When making bivalent or bispecific antibody fragments through the ‘diabody’ approach (Holliger et al., (1993) and patent application WO 94/09131) or by the ‘double scFv’ approach (Mallender and Voss, 1994 and patent application WO 94/13806), again the V_{H} is linked to a (the corresponding) VL .

The multispecific molecules described above can be made by a number of methods. For example, all specificities can be encoded in the same vector and expressed and assembled in the same host cell. This method is particularly useful where the multispecific molecule is a mAb X mAb, mAb X Fab, Fab X F(ab′)₂ or ligand X Fab fusion protein. Various other methods for preparing bi- or multivalent antibodies are described for example described in U.S. Pat. Nos. 5,260,203; 5,455,030; 4,881,175; 5,132,405; 5,091,513; 5,476,786; 5,013,653; 5,258,498; and 5,482,858.

By using a bispecific or multispecific binding member according to the invention the invention offers several advantages as compared to monospecific/monovalent binding members.

A bispecific/multispecific binding member has a first binding domain capable of specifically recognising and binding a Streptococcus protein, in particular PsaA, whereas the other binding domain(s) may be used for other purposes:

In one embodiment at least one other binding domain is used for binding to a Streptococcus protein, such as binding to another epitope on the same Streptococcus protein as compared to the first binding domain. Thereby specificity for the Streptococcus species may be increased as well as increase of avidity of the binding member.

In another embodiment the at least one other binding domain may be used for specifically binding a mammalian cell, such as a human cell. It is preferred that the at least other binding domain is capable of binding an immunoactive cell, such as a leucocyte, a macrophage, a lymphocyte, a basophilic cell, and/or an eosinophilic cell, in order to increase the effect of the binding member in a therapeutic method. This may be accomplished by establishing that the at least one other binding domain is capable of specifically binding a mammalian protein, such as a human protein, such as a protein selected from any of the cluster differentiation proteins (CD), in particular CD64 and/or CD89. A method for producing bispecific antibodies having CD64 specificity is described in U.S. Pat. No. 60,713,517 to Medarex, Inc.

An “effector cell” as used herein refers to an immune cell which Is a leukocyte or a lymphocyte. Specific effector cells express specific Fc receptors and carry out specific immune functions. For example, monocytes, macrophages, neutrophils, eosinophils, and lymphocytes which express CD89 receptor are involved in specific killing of target cells and presenting antigens to other components of the immune system, or binding to cells that present antigens.

Humanised Antibody Framework

It is not always desirable to use non-human antibodies for human therapy, since the non-human “foreign” epitopes may elicit immune response in the individual to be treated. To eliminate or minimize the problems associated with non-human antibodies, it is desirable to engineer chimeric antibody derivatives, i.e., “humanized” antibody molecules that combine the non-human Fab variable region binding determinants with a human constant region (Fc). Such antibodies are characterized by equivalent antigen specificity and affinity of the monoclonal and polyclonal antibodies described above, and are less immunogenic when administered to humans, and therefore more likely to be tolerated by the individual to be treated.

Accordingly, in one embodiment the binding member has a binding domain carried on a humanised antibody framework, also called a humanised antibody.

Humanised antibodies are in general chimeric antibodies comprising regions derived from a human antibody and regions derived from a non-human antibody, such as a rodent antibody. Humanisation (also called Reshaping or CDR-grafting) is a well-established technique for reducing the immunogenicity of monoclonal antibodies (mAbs) from xenogeneic sources (commonly rodent), increasing the homology to a human immunoglobulin, and for improving their activation of the human immune system. Thus, humanized antibodies are typically human antibodies in which some CDR residues and possibly some framework residues are substituted by residues from analogous sites in rodent antibodies.

It is further important that humanized antibodies retain high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional Immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of certain residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is maximized, although it is the CDR residues that directly and most substantially influence antigen binding.

One method for humanising MAbs related to production of chimeric antibodies in which an antigen binding site comprising the complete variable domains of one antibody are fused to constant domains derived from a second antibody, preferably a human antibody. Methods for carrying out such chimerisation procedures are for example described in EP-A-0 120 694 (Celltech Limited), EP-A-0 125 023 (Genentech Inc.), EP-A-0 171 496 (Res. Dev. Corp. Japan), EP-A-0173494 (Stanford University) and EP-A-0 194 276 (Celltech Limited). A more complex form of humanisation of an antibody involves the re-design of the variable region domain so that the amino acids constituting the non-human antibody binding site are integrated into the framework of a human antibody variable region (Jones et al., 1986).

The humanized antibody of the present invention may be made by any method capable of replacing at least a portion of a CDR of a human antibody with a CDR derived from a non-human antibody. Winter describes a method which may be used to prepare the humanized antibodies of the present invention (UK Patent Application GB 2188638A, filed on Mar. 26, 1987), the contents of which is expressly incorporated by reference. The human CDRs may be replaced with non-human CDRs using oligonucleotide site-directed mutagenesis as described in the examples below.

As an example the humanized antibody of the present invention may be made as described In the brief explanation below. The humanized antibodies of the present invention may be produced by the following process:
(a) constructing, by conventional techniques, an expression vector containing an operon with a DNA sequence encoding an antibody heavy chain in which the CDRs and such minimal portions of the variable domain framework region that are required to retain antibody binding specificity are derived from a non-human immunoglobulin, and the remaining parts of the antibody chain are derived from a human immunoglobulin, thereby producing the vector of the invention;
(b) constructing, by conventional techniques, an expression vector containing an operon with a DNA sequence encoding a complementary antibody light chain in which the CDRs and such minimal portions of the variable domain framework region that are required to retain donor antibody binding specificity are derived from a non-human immunoglobulin, and the remaining parts of the antibody chain are derived from a human immunoglobulin, thereby producing the vector of the invention;
(c) transfecting the expression vectors into a host cell by conventional techniques to produce the transfected host cell of the invention; and
(d) culturing the transfected cell by conventional techniques to produce the humanised antibody of the invention.

The host cell may be cotransfected with the two vectors of the invention, the first vector containing an operon encoding a light chain derived polypeptide and the second vector containing an operon encoding a heavy chain derived polypeptide. The two vectors contain different selectable markers, but otherwise, apart from the antibody heavy and light chain coding sequences, are preferably identical, to ensure, as far as possible, equal expression of the heavy and light chain polypeptides. Alternatively, a single vector may be used, the vector including the sequences encoding both the light and the heavy chain polypeptides. The coding sequences for the light and heavy chains may comprise cDNA or genomic DNA or both.

The host cell used to express the altered antibody of the invention may be either a bacterial cell such as Escherichia coli, or a eukaryotic cell. In particular a mammalian cell of a well defined type for this purpose, such as a myeloma cell or a Chinese hamster ovary cell may be used.

The general methods by which the vectors of the invention may be constructed, transfection methods required to produce the host cell of the invention and culture methods required to produce the antibody of the invention from such host cells are all conventional techniques. Likewise, once produced, the humanized antibodies of the invention may be purified according to standard procedures as described below.

Human Antibody Framework

In a more preferred embodiment the invention relates to a binding member, wherein the binding domain is carried by a human antibody framework, i.e. wherein the antibodies have a greater degree of human peptide sequences than do humanised antibodies.

Human mAb antibodies directed against human proteins can be generated using transgenic mice carrying the complete human immune system rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

Such transgenic mice are available from Abgenix, Inc., Fremont, Calif., and Medarex, Inc., Annandale, N.J. It has been described that the homozygous deletion of the antibody heavy-chain joining region (IH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA 90:2551 (1993); Jakobovits et al., Nature 362:255-258 (1993); Bruggermann et al., Year in Immunol. 7:33 (1993); and Duchosal et al. Nature 355:258 (1992). Human antibodies can also be derived from phage-display libraries (Hoogenboom et al., J. Mol. Biol. 227: 381 (1991); Marks et al., J. Mol. Biol. 222:581-597 (1991); Vaughan, et al., Nature Biotech 14:309 (1996)).

In a preferred embodiment the antibodies are produced by the method described in Example 1.

Fragments

In one embodiment of the invention the binding member is a fragment of an antibody, preferably an antigen binding fragment or a variable region. Examples of antibody fragments useful with the present invention include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments which are capable of cross-linking antigen, and a residual other fragment (which is termed pFc′). Additional fragments can include diabodies, linear antibodies, single-chain antibody molecules, and multispecific antibodies formed from antibody fragments.

The antibody fragments Fab, Fv and scFv differ from whole antibodies in that the antibody fragments carry only a single antigen-binding site. Recombinant fragments with two binding sites have been made in several ways, for example, by chemical cross-linking of cysteine residues introduced at the C-terminus of the VH of an Fv (Cumber et al., 1992), or at the C-terminus of the VL of an scFv (Pack and Pluckthun, 1992), or through the hinge cysteine residues of Fab's (Carter et al., 1992).

Preferred antibody fragments retain some or essential all the ability of an antibody to selectively binding with its antigen or receptor. Some preferred fragments are defined as follows:
(1) Fab is the fragment that contains a monovalent antigen-binding fragment of an antibody molecule. A Fab fragment can be produced by digestion of whole antibody with the enzyme papain to yield an intact light chain and a portion of one heavy chain.
(2) Fab′ is the fragment of an antibody molecule and can be obtained by treating whole antibody with pepsin, followed by reduction, to yield an intact light chain and a portion of the heavy chain. Two Fab′ fragments are obtained per antibody molecule. Fab′ fragments differ from Fab fragments by the addition of a few residues at the carboxyl terminus of the heavy chain CH1 domain including one or more cysteines from the antibody hinge region.
(3) (Fab′)₂ is the fragment of an antibody that can be obtained by treating whole antibody with the enzyme pepsin without subsequent reduction. F(ab′)₂ is a dimer of two Fab′ fragments held together by two disulfide bonds.
(4) Fv is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

In one embodiment of the present invention the antibody is a single chain antibody (“SCA”), defined as a genetically engineered molecule containing the variable region of the light chain, the variable region of the heavy chain, linked by a suitable polypeptide linker as a genetically fused single chain molecule. Such single chain antibodies are also refered to as “single-chain Fv” or “sFv” antibody fragments. Generally, the Fv polypeptide further comprises a polypeptide linker between the V_{H} and VL domains that enables the sFv to form the desired structure for antigen binding.

The antibody fragments according to the Invention may be produced in any suitable manner known to the person skilled In the art. Several microbial expression systems have already been developed for producing active antibody fragments, e.g. the production of Fab in various hosts, such as E. coli (Better et al., 1988, Skerra and Pluckthun, 1988, Carter et al., 1992), yeast (Horwitz et al., 1988), and the filamentous fungus Trichoderma reesel (Nyyssonen et al., 1993) has been described. The recombinant protein yields in these alternative systems can be relatively high (1-2 g/l for Fab secreted to the periplasmic space of E. coli in high cell density fermentation, see Carter et al., 1992), or at a lower level, e.g. about 0.1 mg/l for Fab in yeast in fermenters (Horwitz et al., 1988), and 150 mg/l for a fusion protein CBHI-Fab and 1 mg/l for Fab in Trichoderma in fermenters (Nyyssonen et al., 1993) and such production is very cheap compared to whole antibody production in mammalian cells (hybridoma, myeloma, CHO).

The fragments can be produced as Fab's or as Fv's, but additionally it has been shown that a VH and a VL can be genetically linked in either order by a flexible polypeptide linker, which combination is known as an scFv.

Isolated Nucleic Acid Molecule/Vector/Host Cell

In one aspect the invention relates to an isolated nucleic acid molecule encoding at least a part of the binding member as defined above. In one embodiment the nucleic acid molecule encodes a light chain and another nucleic acid encodes a heavy chain. The two nucleic acid molecule may be separate or they may be fused into one nucleic acid molecule, optionally spaced apart by a linker sequence. In particular in relation to antibody fragments the nucleic acid molecule may encode the whole binding member, however dependant on the design of the binding member this may also be relevant for some larger binding members. The nucleic acid molecule preferably is a DNA sequence, more preferably a DNA sequence comprising in its upstream end regulatory elements promoting the expression of the binding member once the nucleic acid molecule is arranged In a host cell.

Accordingly, In one embodiment the Invention relates to a polynucleotide selected from the group consisting of
i) a polynucleotide comprising a sequence selected from the nucleotide sequence of FIG. 16a, FIG. 16b, FIG. 17a, FIG. 17b, FIG. 18a, and FIG. 18b,
ii) a polynucleotide encoding a binding member comprising one or more of the amino acid sequence selected from the group of FIG. 16a, FIG. 16b, FIG. 17a, FIG. 17b, FIG. 18a, and FIG. 18b,
iii) a polynucleotide encoding a fragment of a polypeptide encoded by polynucleotides i), wherein said fragment
   a) is capable of recognising an antigen also being recognised by the binding member of ii), and/or
   b) is capable of binding selectively to an antigen, wherein said antigen is also bound selectively by by the binding member of ii), and/or
   c) has a substantially similar or higher binding affinity to PsaA as a binding domain comprising a predetermined sequence, such as SEQ ID NO 8, SEQ ID NO 16, SEQ ID NO 24, SEQ ID NO 32, SEQ ID NO 40, SEQ ID NO 48,
iv) a polynucleotide, the complementary strand of which hybridizesm under stringent conditions, with a polynucleotide as defined in any of i), ii), iii), and encodes a polypeptide as defined in iii),
v) a polynucleotide comprising a nucleotide sequence which is degenerate to the nucleotide sequence of a polynucleotide as defined in any of i)-iv),

and the complementary strand of such a polynucleotide.

The Invention further relates to a vector comprising the nucleic acid molecule as defined above, either one vector per nucleic acid, or two or more nucleic acids in the same vector. The vector preferably comprises a nucleotide sequence which regulates the expression of the antibody encoded by the nucleic acid molecule.

In yet another aspect the invention relates to a host cell comprising the nucleic acid molecule as defined above.

Also, the invention relates to a cell line engineered to express the binding member as defined above, this cell line for example being a hybridoma of a murine lymphocyte and an immortalised cell line. The cell line may be any suitable cell line, however the cell line P3 is preferred. In another embodiment a CHO cell line is preferred.

Purification of Binding Members

After production the binding members according to the invention are preferably purified. The method of purification used is dependent upon several factors including the purity required, the source of the antibody, the intended use for the antibody, the species in which the antibody was produced, the class of the antibody and, when the antibody is a monoclonal antibody, the subclass of the antibody.

Any suitable conventional methods of purifying polypeptides comprising antibodies include precipitation and column chromatography and are well known to one of skill in the purification arts, including cross-flow filtration, ammonium sulphate precipitation, affinity column chromatography, gel electrophoresis and the like may be used.

The method of purifying an antibody with an anti-immunoglobulin antibody can be either a single purification procedure or a sequential purification procedure. Methods of single and sequential purification are well known to those in the purification arts. In a single-step purification procedure, the antibody is specifically bound by a single anti-immunoglobulin antibody. Non-specifically bound molecules are removed in a wash step and the specifically bound molecules are specifically eluted. In a sequential purification procedure, the antibody is specifically bound to a first anti-immunoglobulin antibody, non-specifically bound molecules are removed in a wash step, and the specifically bound molecules are specifically eluted. The eluant from the first anti-immunoglobulin antibody is then specifically bound to a second anti-immunoglobulin antibody. The non-specifically bound molecules are removed in a wash step, and the specifically bound molecules are specifically eluted. In a preferred embodiment, the antibody is sequentially purified by a first and second anti-immunoglobulin antibody selected from the group consisting of antibodies which specifically bind heavy and light chain constant regions.

A commonly used method of purification is affinity chromatography in which the antibody to be purified is bound by protein A, protein G or by an anti-immunoglobulin antibody. Another method of affinity chromatography, which is well known to those of skill in the art, is the specific binding of the antibody to its respective antigen.

In particular for purifying a multispecific, including a bispecific antibody, a sequential purification procedure may be used, wherein the bispecific antibody comprising two or more variable domains is specifically bound to a first antigen and then to a second antigen.

In an alternative embodiment, a bispecific antibody comprising two or more variable regions is purified by sequential purification by specifically binding the antibody to a first antigen in a first purification step and to a second antigen in a second purification step.

The method of purifying an antibody with an anti-immunoglobulin antibody can be either a single purification procedure or a sequential purification procedure. Methods of single and sequential purification are well known to those in the purification arts. In a single-step purification procedure, the antibody is specifically bound by a single anti-immunoglobulin antibody. Non-specifically bound molecules are removed in a wash step and the specifically bound molecules are specifically eluted. In a sequential purification procedure, the antibody is specifically bound to a first anti-immunoglobulin antibody, non-specifically bound molecules are removed in a wash step, and the specifically bound molecules are specifically eluted. The eluant from the first anti-immunoglobulin antibody is then specifically bound to a second anti-immunoglobulin antibody. The non-specifically bound molecules are removed in a wash step, and the specifically bound molecules are specifically eluted. In a preferred embodiment, the antibody is sequentially purified by a first and second anti-immunoglobulin antibody selected from the group consisting of antibodies which specifically bind heavy and light chain constant regions. In a more preferred embodiment, the antibody is sequentially purified by a first and second anti-immunoglobulin antibody selected from the group consisting of antibodies which specifically bind the heavy chain constant region of IgG and light chain constant regions of kappa and lambda. In an even more preferred embodiment, the anti-immunoglobulin antibody is selected from the group consisting of antibodies which specifically bind the light chain constant regions of kappa and lambda.

Diagnostic Methods

The present invention also describes a diagnostic system, preferably in kit form, for assaying for the presence of Streptococcus, in particular Streptococcus pneumoniae, in a biological sample where it is desirable to detect the presence, and preferably the amount, of bacteria in a sample according to the diagnostic methods described herein.

The diagnostic system includes, in an amount sufficient to perform at least one assay, a binding member composition according to the present invention, preferably as a separately packaged reagent, and more preferably also instruction for use.

The biological sample can be a tissue, tissue extract, fluid sample or body fluid sample, such as blood, plasma or serum.

Packaged refers to the use of a solid matrix or material such as glass, plastic (e.g., polyethylene, polypropylene or polycarbonate), paper, foil and the like capable of holding within fixed limits a binding member of the present invention. Thus, for example, a package can be a glass vial used to contain milligram quantities of a contemplated labelled binding member preparation, or it can be a microtiter plate well to which microgram quantities of a contemplated binding member has been operatively affixed, i.e., linked so as to be capable of binding a ligand.

“Instructions for use” typically include a tangible expression describing the reagent concentration or at least one assay method parameter such as the relative amounts of reagent and sample to be admixed, maintenance time periods for reagent/sample admixtures, temperature, buffer conditions and the like.

A diagnostic system of the present invention preferably also includes a label or indicating means capable of signaling the formation of a binding reaction complex containing a binding member complexed with the preselected ligand.

Any label or indicating means can be linked to or incorporated in an expressed polypeptide, or phage particle that is used in a diagnostic method. Such labels are themselves well-known in clinical diagnostic chemistry.

The labeling means can be a fluorescent labeling agent that chemically binds to antibodies or antigens without denaturing them to form a fluorochrome (dye) that is a useful immunofluorescent tracer. Suitable fluorescent labeling agents are fluorochromes such as fluorescein isocyanate (FIC), fluorescein isothiocyante (FITC), 5-dimethylamine-1-naphthalenesulfonyl chloride (DANSC), tetramethylrhodamine isothiocyanate (TRITC), lissamine, rhodamine 8200 sulphonyl chloride (RB 200 SC) and the like. A description of immunofluorescence analysis techniques is found in DeLuca, “Immunofluorescence Analysis”, in Antibody As a Tool, Marchalonis, et al., eds., John Wiley & Sons, Ltd., pp. 189-231 (1982), which is incorporated herein by reference.

In preferred embodiments, the indicating group Is an enzyme, such as horseradish peroxidase (HRP), glucose oxidase, or the like. In such cases where the principal indicating group is an enzyme such as HRP or glucose oxidase, additional reagents are required to visualize the fact that a receptor-ligand complex (immunoreactant) has formed. Such additional reagents for HRP include hydrogen peroxide and an oxidation dye precursor such as diaminobenzidine. An additional reagent useful with glucose oxidase is 2,2′-amino-di-(3-ethyl-benzthiazoline-G-sulfonic acid) (ABTS).

Radioactive elements are also useful labeling agents and are used illustratively herein. An exemplary radiolabeling agent is a radioactive element that produces gamma ray emissions. Elements which themselves emit gamma rays, such as ¹²⁴I, ¹²⁵I, ¹²⁸I, ¹³²I and ⁵¹Cr represent one class of gamma ray emission-producing radioactive element indicating groups. Particularly preferred is, ¹²⁵I. Another group of useful labeling means are those elements such as ¹¹C, ¹⁸F, ¹⁵O and ¹³N which themselves emit positrons. The positrons so emitted produce gamma rays upon encounters with electrons present in the animal's body. Also useful is a beta emitter, such as ¹¹¹indium or ³H.

The linking of labels, i.e., labeling of, polypeptides and proteins or phage is well known in the art. For instance, proteins can be labelled by metabolic incorporation of radioisotope-containing amino acids provided as a component in the culture medium. See, for example, Galfre et al., Meth. Enzymol., 73:3-46 (1981). The techniques of protein conjugation or coupling through activated functional groups are particularly applicable. See, for example, Aurameas, et al., Scand. J. Immunol., Vol. 8 Suppl. 7:7-23 (1978), Rodwell et al., Biotech., 3:889-894 (1984), and U.S. Pat. No. 4,493,795.

The diagnostic systems can also include a specific binding agent, preferably as a separate package. A “specific binding agent” is a molecular entity capable of selectively binding a binding member species of the present invention or a complex containing such a species, but is not itself a binding member of the present invention. Exemplary specific binding agents are antibody molecules, complement proteins or fragments thereof, S. aureus protein A, and the like. Preferably the specific binding agent binds the binding member species when that species is present as part of a complex.

In preferred embodiments, the specific binding agent is labelled. However, when the diagnostic system includes a specific binding agent that is not labelled, the agent is typically used as an amplifying means or reagent. In these embodiments, the labelled specific binding agent is capable of specifically binding the amplifying means when the amplifying means is bound to a reagent species-containing complex.

The diagnostic kits of the present invention can be used in an “ELISA” format to detect the quantity of a preselected ligand in a fluid sample. “ELISA” refers to an enzyme-linked immunosorbent assay that employs an antibody or antigen bound to a solid phase and an enzyme-antigen or enzyme-antibody conjugate to detect and quantify the amount of an antigen present in a sample and is readily applicable to the present methods.

Thus, in some embodiments, a binding member of the present Invention can be affixed to a solid matrix to form a solid support that comprises a package in the subject diagnostic systems.

A reagent is typically affixed to a solid matrix by adsorption from an aqueous medium although other modes of affixation applicable to proteins and polypeptides can be used that are well known to those skilled in the art. Exemplary adsorption methods are described herein.

Useful solid matrices are also well known in the art. Such materials are water insoluble and include the cross-linked dextran available under the trademark SEPHADEX from Pharmacia Fine Chemicals (Piscataway, N.J.); agarose; beads of polystyrene beads about 1 micron to about 5 millimeters in diameter available from Abbott Laboratories of North Chicago, Ill.; polyvinyl chloride, polystyrene, cross-linked polyacrylamide, nitrocellulose- or nylon-based webs such as sheets, strips or paddles; or tubes, plates or the wells of a microtiter plate such as those made from polystyrene or polyvinylchloride.

The binding member species, labelled specific binding agent or amplifying reagent of any diagnostic system described herein can be provided in solution, as a liquid dispersion or as a substantially dry power, e.g., in lyophilized form. Where the indicating means is an enzyme, the enzyme's substrate can also be provided in a separate package of a system. A solid support such as the before-described microtiter plate and one or more buffers can also be included as separately packaged elements in this diagnostic assay system.

Diagnostic Methods

The present invention also contemplates various assay methods for determining the presence, and preferably amount, of a Streptococcus, in particular Streptococcus pneumoniae, typically present In a biological sample.

Accordingly, the present invention relates to a method of detecting or diagnosing a disease or disorder associated with Pneumococcus in an individual comprising
providing a biological sample from said individual
adding at least one binding member as defined above to said biological sample,
detecting binding members bound to said biological sample, thereby detecting or diagnosing the disease or disorder.

The bound binding members may be detected either directly or indirectly, to the amount of the Streptococcus in the sample.

Those skilled in the art will understand that there are numerous well known clinical diagnostic chemistry procedures in which a binding reagent of this invention can be used to form an binding reaction product whose amount relates to the amount of the ligand in a sample. Thus, while exemplary assay methods are described herein, the invention is not so limited.

Various heterogenous and homogeneous protocols, either competitive or noncompetitive, can be employed in performing an assay method of this invention.

Binding conditions are those that maintain the ligand-binding activity of the receptor. Those conditions include a temperature range of about 4 to 50 degrees Centigrade, a pH value range of about 5 to 9 and an ionic strength varying from about that of distilled water to that of about one molar sodium chloride.

The detecting step can be directed, as is well known in the immunological arts, to either the complex or the binding reagent (the receptor component of the complex). Thus, a secondary binding reagent such as an antibody specific for the receptor may be utilized.

Alternatively, the complex may be detectable by virtue of having used a labelled receptor molecule, thereby making the complex labelled. Detection in this case comprises detecting the label present in the complex.

A further diagnostic method may utilize the multivalency of a binding member composition of one embodiment of this invention to cross-link ligand, thereby forming an aggregation of multiple ligands and polypeptides, producing a precipitable aggregate. This embodiment is comparable to the well-known methods of immune precipitation. This embodiment comprises the steps of admixing a sample with a binding member composition of this invention to form a binding admixture under binding conditions, followed by a separation step to isolate the formed binding complexes. Typically, isolation is accomplished by centrifugation or filtration to remove the aggregate from the admixture. The presence of binding complexes indicates the presence of the preselected ligand to be detected.

Pharmaceutical Compositions

In a preferred aspect the present invention contemplates pharmaceutical compositions useful for practising the therapeutic methods described herein. Pharmaceutical compositions of the present invention contain a physiologically tolerable carrier together with at least one species of binding member as described herein, dissolved or dispersed therein as an active ingredient. In a preferred embodiment, the pharmaceutical composition is not immunogenic when administered to a human individual for therapeutic purposes, unless that purpose is to induce an immune response.

In one aspect the invention relates to a pharmaceutical composition comprising at least one binding member as defined above. In a preferred embodiment the pharmaceutical composition comprises at least two different binding members as defined above in order to increase the effect of the treatment.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects such as nausea, dizziness, gastric upset and the like.

The preparation of a pharmacological composition that contains active ingredients dissolved or dispersed therein is well understood in the art. Typically such compositions are prepared as sterile injectables either as liquid solutions or suspensions, aqueous or non-aqueous, however, solid forms suitable for solution, or suspensions, in liquid prior to use can also be prepared. The preparation can also be emulsified.

The active ingredient can be mixed with exciplents which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like, which enhance the effectiveness of the active ingredient.

The pharmaceutical composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, propylene glycol, polyethylene glycol and other solutes.

Liquid compositions can also contain liquid phases in addition to and to the exclusion of water. Exemplary of such additional liquid phases are glycerin, vegetable oils such as cottonseed oil, organic esters such as ethyl oleate, and water-oil emulsions.

A pharmaceutical composition contains a binding member of the present invention, typically an amount of at least 0.1 weight percent of antibody per weight of total pharmaceutical composition. A weight percent is a ratio by weight of antibody to total composition. Thus, for example, 0.1 weight percent Is 0.1 grams of antibody per 100 grams of total composition.

The invention also relates to a method for preparing a medicament or pharmaceutical composition comprising an antibody of the invention, the medicament being used for immunotherapy of a disease or disorder associated with Streptococcus, in particular Streptococcus pneumoniae, such as pneumonia, meningitis and sepsis, comprising admixing at least one binding member as defined above with a physiologically acceptable carrier.

Furthermore, the invention relates to the use of a binding member as defined above for the production of a pharmaceutical composition for the treatment of a disease or disorder associated with Streptococcus, in particular Streptococcus pneumoniae, such as pneumonia, meningitis and sepsis.

The pharmaceutical composition may also be a kit-in-part further including an antibiotic agent, such as antibiotics selected from β-lactams, cephalosporins, penicilins and aminoglycosides, and/or include an immunostimulating agent, such as cytokines, interferons, growth factors, for example GCSF or GM-CSF. The kit-in-part may be used for simultaneous, sequential or separate administration.

Furthermore, the pharmaceutical composition may include the binding member according to the invention in combination with the Streptococcus protein PsaA, in particular as a vaccine. It has been found that by combining the binding member according to the invention with the protein PsaA, the immunising properties of the combination product is better than for the protein PsaA alone. This may be due to the fact that the protein PsaA is presented to the immune system by the binding member.

Therapeutic Methods

The binding members according to the present invention are particular useful in therapeutic methods due to their high affinity and specificity. Accordingly, the binding members can be used immunotherapeutically towards a disease or disorder associated with Streptococcus, in particular Streptococcus pneumoniae, such as pneumonia, meningitis and sepsis.

The term “immunotherapeutically” or “immunotherapy” as used herein in conjunction with the binding members of the invention denotes both prophylactic as well as therapeutic administration. Thus, the binding members can be administered to high-risk patients in order to lessen the likelihood and/or severity of disease, administered to patients already evidencing active infection, or administered to patients at risk of infection.

The dosage ranges for the administration of the binding members of the invention are those large enough to produce the desired effect in which the symptoms of the disease are ameliorated or the likelihood of infection decreased. Generally, the dosage will vary with the age, condition, sex and extent of the disease in the patient and can be determined by one of skill in the art. The dosage can be adjusted by the individual physician in the event of any complication.

A therapeutically effective amount of an binding member of this invention is typically an amount of antibody such that when administered in a physiologically tolerable composition is sufficient to achieve a plasma concentration of from about 0.1 microgram (μg) per milliliter (ml) to about 100 μg/ml, preferably from about 1 μg/ml to about 5 μg/ml, and usually about 5 μg/ml. Stated differently, the dosage can vary from about 0.1 mg/kg to about 300 mg/kg, preferably from about 0.2 mg/kg to about 200 mg/kg, most preferably from about 0.5 mg/kg to about 20 mg/kg, in one or more dose administrations daily, for one or several days.

The binding members of the invention can be administered parenterally by injection or by gradual infusion over time. Although the infection may be systemic and therefore most often treated by intravenous administration of pharmaceutical compositions, other tissues and delivery means are contemplated where there is a likelihood that targeting a tissue will result in a lessening of the disease. Thus, antibodies of the invention can be administered parenterally, such as intravenously, intraperitoneally, intramuscularly, subcutaneously, intracavity, transdermally, and can be delivered by peristaltic means.

The pharmaceutical compositions containing a binding member of this invention are conventionally administered intravenously, as by injection of a unit dose, for example. The term “unit dose” when used in reference to a pharmaceutical composition of the present invention refers to physically discrete units suitable as unitary dosage for the subject, each unit containing a predetermined quantity of active material calculated to produce the desired therapeutic effect In association with the required diluent; i.e., carrier, or vehicle.

The therapeutic method may further include the use of a kit-in-part as defined above.

### EXAMPLES

The invention is further explained through the examples below; the examples are not to be construed as limiting to the invention.

### Example 1

Production of Anti-PsaA Antibodies

Mice having the ability of making fully human antibodies were used. The mice were HuMAb-Mouse, obtained from Medarex, Inc.

His6-psaA protein (wherein the PsaA protein has the sequence depicted in FIG. 15) was used as antigen for the immunization experiments. The concentrations of these proteins were calculated by spectrophotometer, and their purity was ascertained by SDS-PAGE after silver staining. Proteins were prepared for immunization using complete Freund's, incomplete Freund's, and RIBI as an adjuvant, as appropriate. Groups of 12 to 18 male HuMAb mice were used for all immunization experiments. Each mouse was immunized by Intraperitoneal (i.p.) and subcutaneous injection of 20-50 μg of His6-psaA (PsaA amino acid sequence is shown in FIG. 15) at three-week intervals. Some mice received 5×10e8 heat-inactivated Streptococcus pneumoniae R6 cells i.p. Sera were collected from mice by retro-orbital bleeding. Titers to rpsaA were determined by ELISA. Mice were boosted intravenously prior to sacrifice.

8 fusions were performed and resulted in 65 viable cell clones. These were subcloned and 42 positive clones identified by the method described in 1a below were selected for further research. Among these 26 were selected and monoclonal antibodies purified. 3 were discarded because of poor reactivity; thus 23 clones were evaluated. Among the 23 clones a prioritized list of candidate MAbs was established. The four top-prioritized were further evaluated in an in vivo model.

1a) Identification of Streptococcus pneumoniae R6 Binding Antibodies

Hydridoma supernatants are tested in 3 dilutions against S. pneumococus R6 in a sandwich enzyme linked immunosorbent assay with reagent excess.

Devices:

ELISA reader, BIO-TEK EL 800

ELISA washer, handheld model

Incubator 37° C.

Pipettes

Materials:

Tips

Reagent tray

Plate cover

96-well microtiterplate (Nunc Maxisorp)

Bacteria:

S. pneumococcus R6 (SSI, Pneumokoklaboratoriet) from frozen stock with a concentration of 10 e10/ml. Thaw rapidly at 37° C. and dilute 1:100 in coating buffer.

Reagents:

Na2CO3, pH 9.6

PBS, pH 7.4

Tween 20,

Rabbit-anti-Human IgA, G, M HRP-conjugated, DAKO #P212

Rabbit-anti-Mouse Ig HRP-conjugated, DAKO #P260

Swine-anti-Rabbit Ig HRP-conjugated, DAKO #P217

0.1 M Citric Acid, pH 5.0

OPD 10 mg tablets, Kem-En-Tec no. 4260

H2O2

H2SO4

Coating Buffer: Na2CO3 0.05 M, pH 9:6, expire two weeks after production, storage at4° C.

Dilution and wash buffer: PBS with 0.1% Tween20, expire one week after production, storage at 4° C.

Secondary Antibody: Dilute 1:1500 in dilution buffer, freshly made every day.

Substrate: Dissolve 1 OPD tablet in 10 ml Citric Acid and add 10 μl H2O2. Freshly made every day.

Stop reagent: 1.2 M H2SO4, expire 1 year after production, storage at RT.

Controls:

Blank: PBS with 0.1% Tween 20

Positive controls: Affinity purified Ra-a-PsaA

Samples:

Hybridoma culture supernatants diluted 1:2, 1:50 and 1:1250 in dilution buffer.

Procedure:

100 μL bacteria per well (=10 e7) are coated into 96-microwells and the plate is incubated overnight at 4° C. Discard excess fluid. The plate is washed three times with 300 μL wash buffer and can hereafter be stored at 4° C. for a period of 1 month. 100 μL blank, positive control or sample is added and the plate is incubated 2 hours at 37° C. The plate is washed three times with 300 μL wash buffer 100 μL secondary antibody is added and the plate is incubated 1 hour at 37° C. The plate is washed three times with 300 μL wash buffer. The plate is washed two times with 300 μL Dem. H₂O.100 μL substrate is added and the plate is incubated 30 min at RT. 100 μL stop reagent is added and the plate is read at A490 nm.

### EXAMPLE 2

In vitro Effect of Monoclonal Anti-PsaA Antibodies

The monoclonal antibodies obtained in Example 1 were tested in vitro against Pneumococcus of various strains identifying antibodies against whole bacteria (ELISA) see method 1, and using Western blot, see method 2, below.

Method 2

Identification of Bacteria Binding Antibodies—Western Blot.

First bacterial proteins are transferred from NuPAGE Tris-Bls Gels to polyvinylidene difluoride (PVDF) membranes by the following method.

Samples:

Electrophoresed NuPAGE Tris-Bis Gels

Devices:

XCell Surelock™ Mini-Cell with Blot Module

Power supply

Materials:

Pre-cut blotting PVDF membrane (0.45 μm) and filter papers (Invitrogen #LC2005)

Pipettes

Tips

Trays

Reagents:

NuPAGE Transfer Buffer (Invitrogen #NP0006)

Milli-Q grade H₂O

Ethanol, 96%

Procedure:

Transferring One Gel

a) 500 ml of Transfer Buffer was prepared by adding 25 ml 20X NuPAGE® Transfer Buffer and 100 ml Ethanol 96% to 375 ml Milli-Q grade H₂O. Blotting pads were soaked in 350 ml of Transfer Buffer. PVDF membrane was soaked in 96% Ethanol for 30 sec. and wash in transfer buffer for 2 min, and the filter paper was briefly soaked in Transfer Buffer.

A piece of pre-soaked filter paper was placed on top of the gel (adhered to the bottom plate) and any trapped air bubbles were removed.

The plate was turned over so the gel and filter paper were facing downwards over a gloved hand or clean flat surface. The pre-soaked transfer membrane was placed on the gel and trapped air bubbles removed.

Another pre-soaked filter paper was placed on top of the membrane, and any air bubbles were removed.

b) Two soaked blotting pads ware placed into the cathode (−) core of the blot module. The gel/membrane assembly was carefully picked up and placed on blotting pad in the correct orientation, so the gel is closest to the cathode core. Two soaked blotting pads are placed on top of the sandwich, and the anode (+) core on top of the pads.

c) The blot module was held together firmly and slid it into the guide rails on the Lower Buffer Chamber. The Gel Tension Wedge was sledged into the Lower Buffer Chamber and the Wedge locked into position.

The blot module was filled with Transfer Buffer until the gel/membrane assembly was covered. The Outer Buffer Chamber was filled with 650 ml Milli-Q grade H₂O, the lid placed on the unit and the electrical leads connected to the power supply. Transfer for PVDF membranes were performed using 30 V constant for 1 hour. The expected start current was 170 mA and end current is 110 mA. Western Blots were developed, and the dried membranes were stored in closed container/pouch at 4° C. for 1 week.

Transferring Two Gels

1. Repeat Steps a) above twice to prepare 2 gel/membrane sandwiches.

2. Place two pre-soaked blotting pads on the cathode core of the blot module.

3. Place the first gel/membrane assembly on the blotting pad in correct orientation, so the gel is closest the cathode core.

4. Add another pre-soaked blotting pad on top of the first membrane assembly.

5. Place the second gel/membrane sandwich on top of the blotting pad in the correct orientation so the gel is closest the cathode core.

6. Proceed with Steps c) from Transferring One Gel.

The identification is conducted as described below:

PVDF membranes with electrophoretically transferred proteins, bacterial lysate(s) or lipopolysaccharide.

Materials:

Tubes

Trays

Pipettes

Tips

Reagents:

WesternBreeze Blocker/Diluent A+B, Invitrogen no. WB7050

Western Breeze Wash Solution (16×), Invitrogen no. WB7003

NBT/BCIP Liquid Substrate, Sigma-Aldrich no. B3679

Simply Blue Safestain, Invitrogen no. LC 6060

Rabbit anti-Human IgG AP, DAKO D0336

Rabbit anti-Mouse Ig AP, DAKO D0314

Swine anti-Rabbit 1g AP, DAKO D0306

Milli-Q grade H2O

Ethanol 96%

Wash buffer:

10 ml Western Breeze Wash Solution (16×)

150 ml Milli-Q grade H2O

Blocking Solution:

5 ml Milli-Q grade H2O

2 ml Blocker/Diluent (Part A)

3 ml Blocker/Diluent (Part B)

Primary Antibody Solution:

7 ml Milli-Q grade H2O

2 ml Blocker/Diluent (Part A)

1 ml Blocker/Diluent (Part B)

Primary antibody to final concentration 0.2-1 μg/ml

Secondary Antibody Solution:

7 ml Milli-Q grade H2O

2 ml Blocker/Diluent (Part A)

1 ml Blocker/Diluent (Part B)

5 μl Secondary antibody (Final dilution 1:2000)

Procedure:

1. Dried PVDF membranes are re-wetted in Ethanol 96% and rinsed two times for 5 min each in 20 ml of Milli-Q grade H2O, proceed to step 4.

2. Freshly blotted membranes are placed on glass plate and MW marker is cut off. MW marker is stained for 15-30 min in Simply Blue SafeStain and de-stained with 30% Ethanol until background is clear. Wash twice in Milli-Q grade H2O.

3. The membrane is washed two times for 5 min each in 20 ml Milli-Q grade H2O.

4. Place membrane in 10 ml of Blocking Solution in covered, plastic dish for 30 min at RT, gently shaking. Decant Blocking Solution

5. Rinse with 20 ml of H2O for 5 min at RT, gently shaking. Decant. Repeat once. Membrane can now be cut into strips if necessary.

6. Incubate membrane with 10 ml of Primary Antibody Solution for 1 hour at RT, gently shaking. Alternatively, incubate o.n. at 4° C.

7. Wash four times for 5 min each time with 20 ml Wash Buffer.

8. Incubate membrane with 10 ml of Secondary Antibody Solution for 30 min at RT.

9. Wash four times for 5 min each time with 20 ml Wash buffer.

10. Rinse the membrane three times for 2 min each time with 20 ml of Milli-Q grade H₂O.

11. Incubate membrane in 5 ml of NBT/BCIP Liquid Substrate until purple bands develop (1-60 min.).

12. Stop development by rinsing the membrane three times for 2 min each time with 20 ml of Milli-Q grade H₂O.

13. Dry the membrane on a clean piece of filter paper.

Results

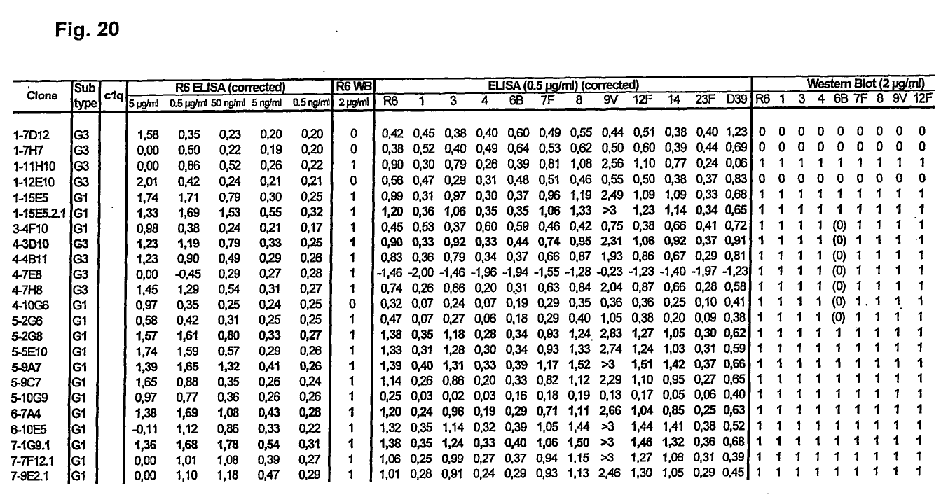

In FIG. 20, the results from the in vitro tests are listed.

### EXAMPLE 3

Measurement of Anti-PsaA Antibody Affinity

The following general description was applied in all affinity measurements.

Instrument and Software Used:
a. BIAcore 3000, surface plasmon resonance instrument
b. BiaEval v3.2, software for data analysis.
C. All running buffers (HBS-EP/HBS-P) from Biacore.

Method to Immobilize Protein G via Amines on a CM5 Chip (Amine Coupling Method)
a. Normalize the chip at least twice with appropriate buffer
b. A 0.5 μg/mL dilution of Protein-G is made in 10 mM sodium acetate buffer of pH 2.9
c. Activate the CM5 chip for 7 minutes, by flowing freshly mixed EDC & NHS at a flow rate of 5 μL/min, according to the method mentioned in Biacore Handbook.
d. Inject Protein-G sample for 22 minutes over this activated surface.
e. Deactivate by flowing 1M ethonolamine-HCl for 10 minutes.
f. This method couples about 10000 RUs of Protein-G on the activated surface.
g. For the blank surface, the same activation and deactivation procedure is followed without the injection of Protein-G.

Method to Immobilize PsaA via Amines on a CM5 Chip (Amine Coupling Method)
a. A CM5 chip was normalized as above
b. PsaA dilutions are made in concentrations ranging from 50 to 150 μg/mL, In sodium 10 mM acetate buffer of pH 4.0
c. The chip is activated for 7 minutes by flowing freshly mixed EDC and NHS.
d. Inject PsaA, made in acetate buffer, by manual injection until the amount captured on the chip reaches the desired level (in our case, 350 and 800 RUs)
e. Deactivate the chip by injecting 1 M ethanolamine-HCl for 10 minutes
f. Regenerate the chip with a mild acid or base to remove unbound/loosely bound molecules from the chip surface.
g. The blank surface is generated in the same method, but without the step of injecting the protein.

Method to Determine the Avidity of Binding of Anti-PsaA Antibodies with PsaA
a. The general methodology followed to measure avidity of antibodies is to flow antibodies of at least five different concentrations over the antigen surface (described above) at high flow rates. The high flow rates are required to minimize the antibody re-binding to the antigen surface. The association and dissociation phases could vary between 5 to 10 minutes and 30 to 40 minutes respectively. Though this experimental design will lead to the measurement of avidities of the antibodies; we try to minimize the effect of this phenomenon in the estimation of avidity. The data analysis was carried out after carefully not including regions that exhibited biphasic behavior of association/dissociation, which was then fit to a 1:1 Langmuir model. The method of selecting the data for analysis ensures that the estimates of avidity are closer to the true affinities.
b. Antibody concentration: 3,2,1,0.75 or 0.5 μg/mL (corresponding to 40.02, 26.68, 13.34, 10.0 & 6.67 nM of binding sites). All dilutions were made in the running buffer, HBS-EP, pH 7.2.
c. Flow rate of 30 μL/min. Association phase 8 min, dissociation phase 30 to 40 min.
d. Regeneration of the surface: flow rate 100 μL/min, buffer: 100 mM HCl w/150 mM NaCl, time: 1-2 minutes.

Experiment 3a) Binding Affinity of 8 Purified Anti-PsaA huMabs

Reagents:

| Name | Conc. | Lot # |
|---|---|---|
| PsaA | 0.6 mg/mL | 021127 |
| 11H10 | 1.24 mg/mL | |
| 12E10 | 1.35 mg/mL | |
| 9C3 | 0.83 mg/mL | |
| 8A12 | 1.33 mg/mL | |
| 4F10 | 1.85 mg/mL | |
| 7H7 | 1.09 mg/mL | |
| 7D12 | 1.61 mg/mL | |
| 6D10 | 0.80 mg/mL | |

Instrument Used: Biacore: 3000
Chip used: Coupling Buffer: 10 mM Acetate, pH 4.0
Coupling Conc: 50-150 ug/mL
Amt. immobilized (Feb. 12, 2003):
Fc1 & 3 = Blank
Fc2 = PsaA: 353RUs
Fc4: PsaA: 824RUs
Amt. immobilized (Feb. 20, 2003):
Fc1 & 3 = Blank
Fc2 = PsaA: 1145RUs
Fc4: PsaA: 355RUs

Experimental Conditions:

| Antibody Conc.: 5, 4, 3, 2 & 1 ug/mL | |
|---|---|
| (66.7, 53.36, 40.02, 26.68 &13.34 nM) | |
| Running buffer: HBS-EP | Flow rate: 30 uL/min |
| Association Time: 8 min. | Dissociation Time: 40 min |
| Regeneration Buffer: 100 mM HCl + 150 mMNaCl | |
| Flow rate: 100 uL/min | Regeneration Time: 1 min |

Results:

| | K_{D} × 10⁻⁹ | kₐ × 10⁴ | k_{d} × 10⁻⁵ |
|---|---|---|---|
| Sample ID | (M) | (1/Ms) | (1/s) |
| 11H10 | 2.45 | 6.86 | 16.8 |
| 12E10 | 0.64 | 7.93 | 5.07 |
| 9C3* | 7.07 | 1.02 | 7.2 |
| 8A12 | Negligible binding at the high | | |
| | density PsaA surface | | |
| 4F10 | 1.33 | 7.67 | 10.2 |
| 7H7 | 1.14 | 6.32 | 7.18 |
| 7D12 | Poor binding at both the low & | | |
| | high density surface | | |
| 6D10 | 0.85 | 13.5 | 11.5 |

*Binding curves at higher conc. did not reach the saturation.

Experiment 3b) Binding Affinity of 3 Purified Anti-PsaA HuMabs

Reagents:

| Name | Conc. | Lot # |
|---|---|---|
| PsaA | 0.36 mg/mL | 064014B |
| 9A7 | 3.83 mg/mL | |
| 1G9 | 0.98 mg/mL | |
| 15E5 | 1.36 mg/mL | |

Instrument Used: Biacore: 3000
Chip used: CM5
Coupling Buffer: 10 mM Acetate, pH 4.0
Coupling Conc: 50-150 ug/mL
Amt. immobilized:
Fc1 & 3 = Blank
Fc2 = PsaA: 353 RUs
Fc4: PsaA: 824 RUs

Experimental Conditions:

| Antibody Conc.: 4, 3, 2, 1 & 0.5 ug/mL | |
|---|---|
| (53.36, 40.02, 26.68, 13.34 & 6.67 nM) | |
| Running buffer: HBS-EP | Flow rate: 25 uL/min |
| Association Time: 5 min. | Dissociation Time: 45 min |
| Regeneration Buffer: 100 mM HCl + 150 mM NaCl | |
| Flow rate: 100 uL/min | Regeneration Time: 1 min |

Results:

| | K_{D} | kₐ | k_{d} |
|---|---|---|---|
| Sample ID | (M) | (1/Ms) | (1/s) |
| 1G9 | 6.34 × 10⁻¹¹ | 4.35 × 10⁵ | 2.87 × 10⁻⁵ |
| 9A7 | 3.74 × 10⁻¹¹ | 3.77 × 10⁵ | 1.41 × 10⁻⁵ |
| 15E5 | 1.18 × 10⁻¹⁰ | 5.71 × 10⁴ | 6.72 × 10⁻⁶ |

Experiment 3c) Binding Affinity of 14 Purified Anti-PsaA HuMabs

Reagents:

| Name | Conc. | Lot # |
|---|---|---|
| PsaA | 0.6 mg/mL | 021127 |
| 3D10 | 3.5 mg/mL | |
| 4B11 | 3.93 mg/mL | |
| 7E8 | 4.42 mg/mL | |
| 7H8 | 3.18 mg/mL | |
| 10G6 | 2.36 mg/mL | |
| 2G6 | 1.94 mg/mL | |
| 2G8 | 5.35 mg/mL | |
| 9C7 | 3.35 mg/mL | |
| 10G9 | 1.07 mg/mL | |
| 5E10 | 3.21 mg/mL | |
| 7A4 | 2.23 mg/mL | |
| 10E5 | 2.07 mg/mL | |
| 7F12 | 0.256 mg/mL | |
| 9E2 | 0.219 mg/mL | |

Instrument Used: Biacore: 3000
Chip used: CM5
Chip prepared on: Feb. 12, 2003
Coupling Buffer: 10 mM Acetate, pH 4.0
Coupling Conc: 50-150 ug/mL
Amt. immobilized (Feb. 12, 2003):
Fc1 & 3 = Blank
Fc2 = PsaA: 353RUs
Fc4: PsaA: 824RUs

Experimental Conditions:

| Antibody Conc.: 3, 2, 1, 0.75 & 0.50 ug/mL (40.02, | |
|---|---|
| 26.68, 13.34, 10.0, 6.67 nM) | |
| Running buffer: HBS-EP | Flow rate: 30 uL/min |
| Association Time: 8 min. | Dissociation Time: 30-40 min |
| Regeneration Buffer: 100 mM HCl + 150 mMNaCl | |
| Flow rate: 100 uL/min | Regeneration Time: 45 sec−1 min |

Results:

| | K_{D} × 10⁻¹⁰ | kₐ × 10⁵ | k_{d} × 10⁻⁵ |
|---|---|---|---|
| Sample ID | (M) | (1/Ms) | (1/s) |
| 3D10 | 1.26 | 2.25 | 2.83 |
| 4B11 | 0.56 | 1.45 | 0.82 |
| 7E8* | 0.78 | 0.66 | 0.51 |
| 7H8 | 1.71 | 1.65 | 2.82 |
| 10G6^{†} | Negligible binding at high | | |
| | density PsaA surface | | |
| 2G6 | 1.75 | 2.07 | 3.61 |
| 2G8* | 1.16 | 0.79 | 0.92 |
| 9C7 | 0.82 | 0.21 | 0.17 |
| 10G9* | 3.14 | 0.35 | 1.1 |
| 5E10* | 1.61 | 0.70 | 0.11 |
| 7A4 | 2.12 | 1.64 | 3.49 |
| 10E5 | 0.39 | 2.2 | 0.86 |
| 7F12 | 0.47 | 5.67 | 2.67 |
| 9E2 | 0.42 | 7.33 | 3.1 |

*Binding curves at higher conc. did not reach the saturation.

### EXAMPLE 4

In vivo Testing of Candidate Antibodies

The effect of anti-PsaA huMabs in the treatment of infection caused by Pneumococcus was determined as described below.

Materials:
Transgenic (human CD64) female mice (8-12 weeks, weight 19-20 g))
0.9% saline (MU)
PBS pH 7.0
5% blood plates
Filtered bovine broth
Monoclonal antibodies:
   anti-PsaA 5-9A7
   anti-PsaA 1-1-15E5
   anti-PsaA 7-1G9
   anti-PsaA 4-3D10

Strains: Pneumococcus D39 (type 2) (F1/S1/E2)

Method

Day-1:
Pn.-strain is seeded on a 4×5% bloodplate, and Incubated overnight at 35° C.

Day 0

The Pneumococcus strain is suspended in filtered broth to 10⁸ CFU/ml (cf. MU/F074-01), and diluted to 1×10⁶ CFU/ml (50 μl 10⁸ CFU/mi i 4.95 ml PBS) and further diluted with antibody (see scheme). The bacteria/antibody mixture is shaken and incubated for 10 min. at 35° and then the mice are inoculated with 0.5 ml i.p.

| | | | Bacteria. | |
|---|---|---|---|---|
| | | | appr. | |
| | | ANTIBODY | 10⁶ | |
| Solution | Final conc. | (500 μg/ml) | CFU/ml | PBS |
| Cage 1 | 10^{5 CFU/ml} | none | 0.6 ml | 5.40 ml |
| Cage 2 | 200 μg/10⁵ CFU/ml | 1.6 ml | 0.4 ml | 2.00 ml |
| Cage 3 | 20 μg/10⁵ CFU/ml | 0.2 ml | 0.5 ml | 4.30 ml |
| Cage 4 | 2 μg/10⁵ CFU/ml | 0.02 ml | 0.5 ml | 4.48 ml |

Scheme

| | | | Mice # | Mice # |
|---|---|---|---|---|
| | | | withdrawn | withdrawn |
| | No. | | time | time |
| Cage | mice | Antibody/bact./mice | 2 hours | 5 hours |
| 1 | 6-9 | PBS/5 × 10^{4 CFU/mice} | 1-2-3 | 4-9 |
| 2 | 6-9 | 40-100 μg/5 × 10⁴ CFU/mice | | 10-15 |
| 3 | 6-9 | 10 μg/5 × 10⁴ CFU/mice | | 16-21 |
| 4 | 6-9 | 1 μg/5 × 10⁴ CFU/mice | | 22-27 |

*) Serum and peritoneal fluid was frozen at −20° C. for antibody concentration measurements

Withdrawal of Blood Samples

The mice were sedated with CO₂. A cut was made in the axilla, and blood collected in tube glass 0.100 μl was transferred to glass 1 and mixed thoroughly, whereafter 100 μl was spread on a bloodplate with a glass rod. Then CFU determination was conducted. The rest of the blood was centrifuged at 2000×G for 7 min. and serum transferred to another tube, stored at −20° C.

Withdrawal of Peritoneal Fluid

The mice were sacrificed, 2 ml sterile saline was injected into the abdomen, and 10 sec. later the fluid was withdrawn with a sterile Pasteur pipette and transferred to an Eppendorf tube. CFU determination was conducted. The rest of the peritoneal fluid was stored at −20° C.

Results

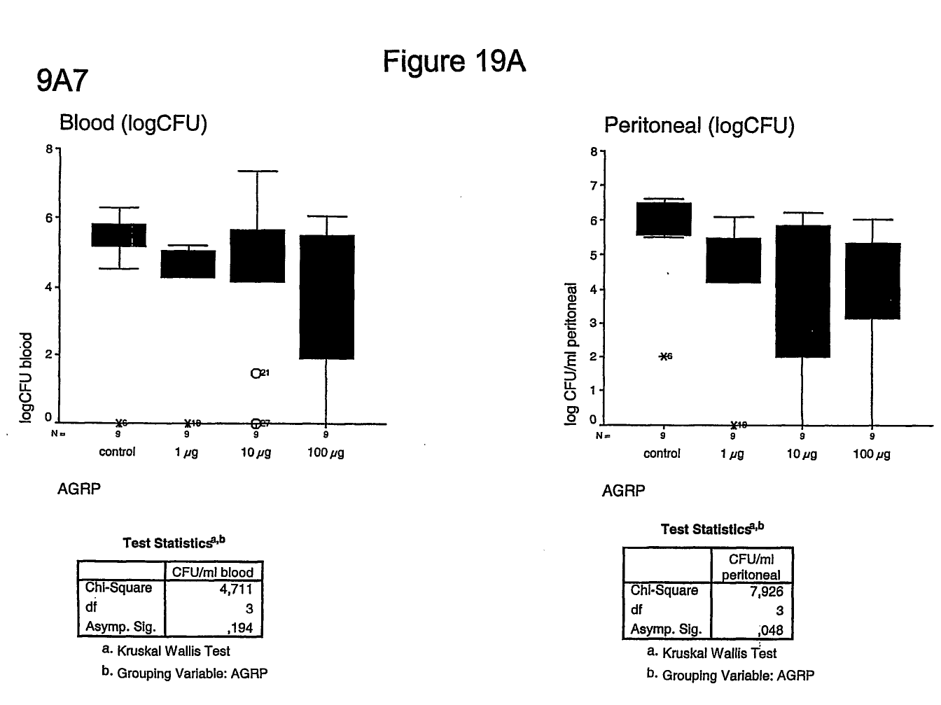

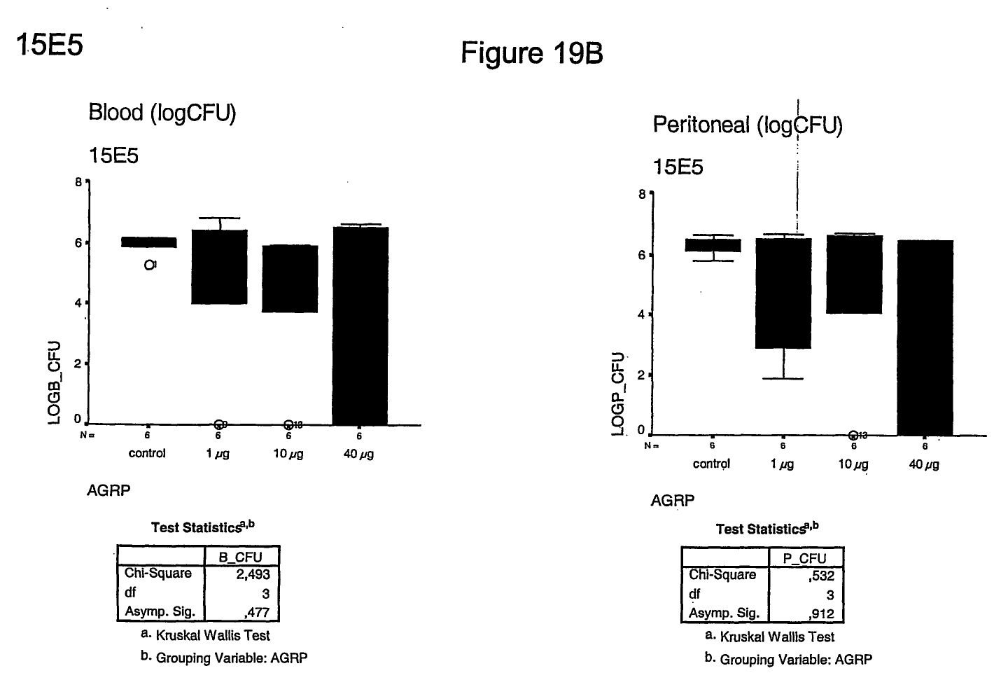

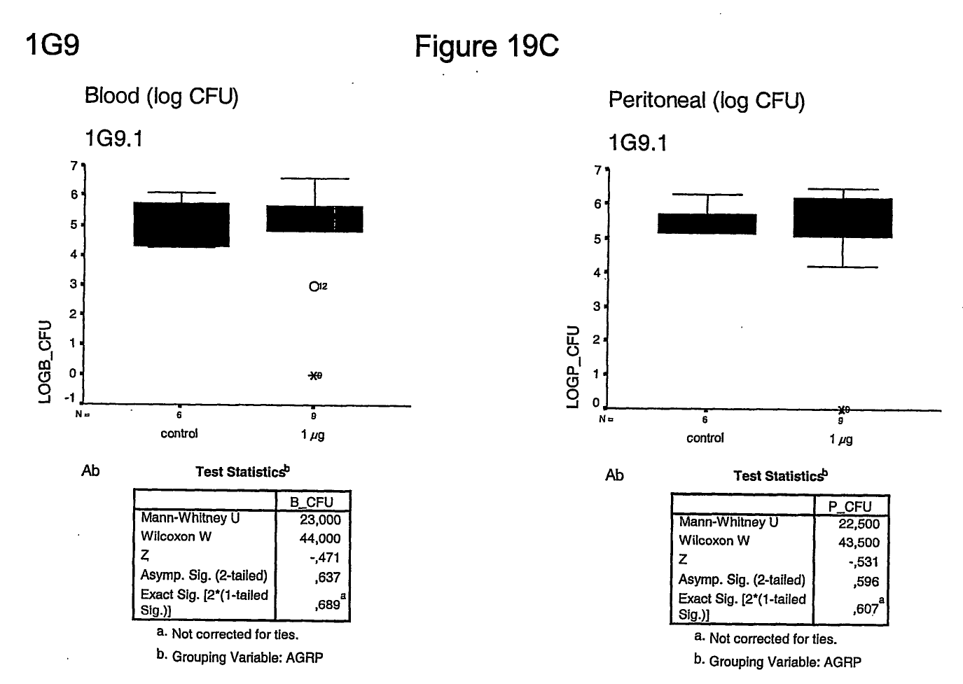

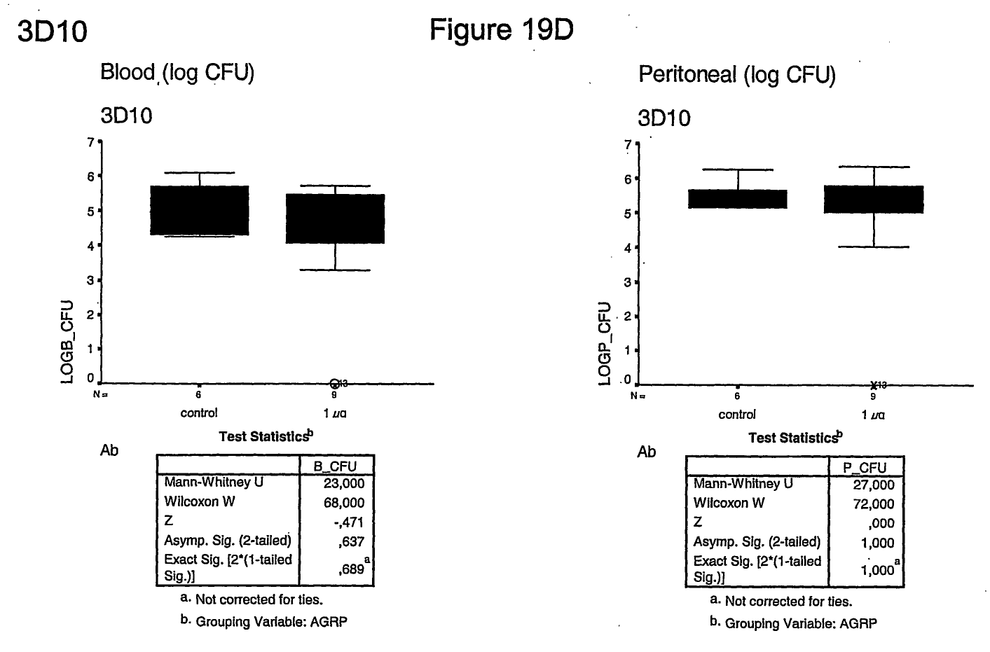

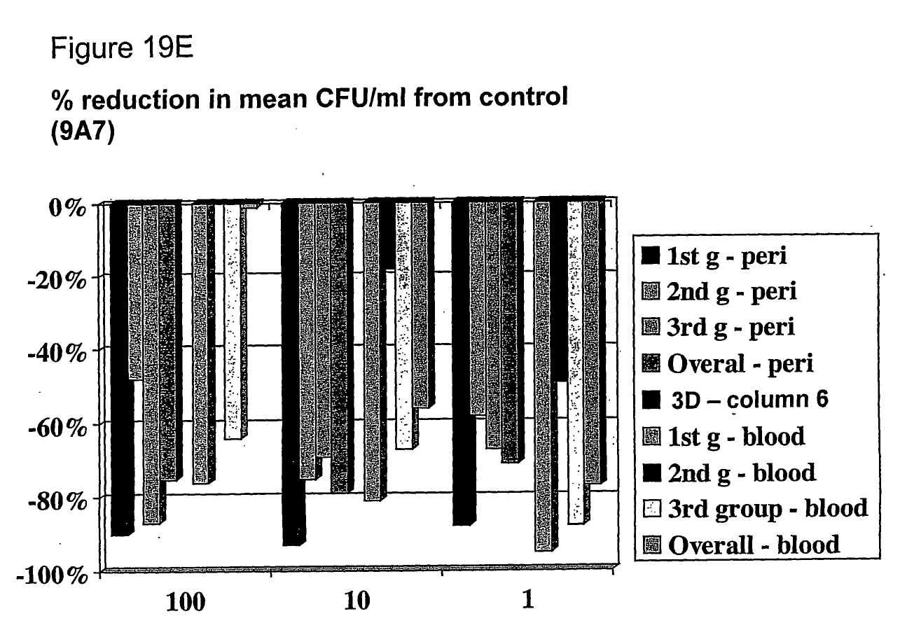

The results are shown in FIG. 19A-E.

### EXAMPLE 5

Generation of Anti-CD64×Anti-PsaA 5-9A7 Bispecific Antibody

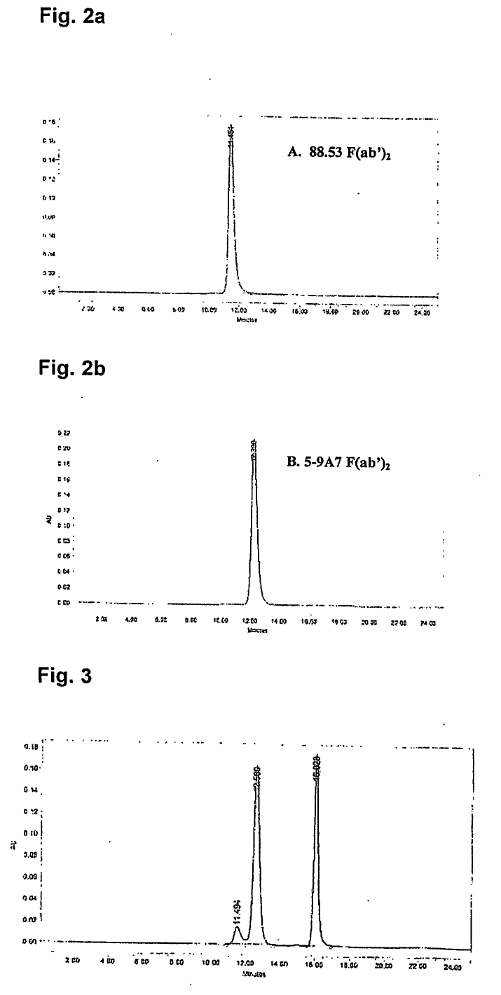

F(ab′)₂ fragments of each of the HuMAbs, anti-CD64 (88.53), and anti-PsaA 5-9A7 were generated by pepsin digestion and purified to homogeneity by Superdex 200 gel filtration chromatography. Size exclusion HPLC was performed and profiles are depicted for each of the F(ab′)₂ in FIG. 2. By this type of analysis both of the F(ab′)₂ fragments were >95% pure.

A Fab′ fragment of the 88.53 was generated by mild reduction of the inter-heavy chain disulfide bonds of the F(ab′)₂ fragment with mercaptoethanolamine (MEA). The exact reducing conditions were determined prior to conjugation in small-scale experiments. Size exclusion HPLC was performed and the profile is depicted for the Fab′ in FIG. 3. By this type of analysis the 88.53 Fab′ was >90% pure.

The Fab′ fragment of the 88.53 was separated from free MEA by G-25 column chromatography. The Fab′ fragment was incubated with dinitrothiobenzoate (DTNB) to generate a Fab-TNB conjugate.

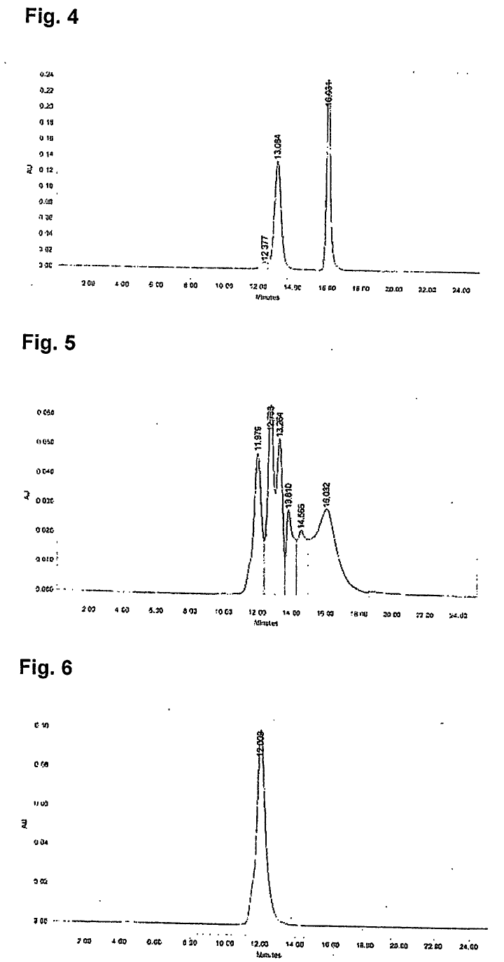

A Fab′ fragment of the 5-9A7 was generated by mild reduction of the inter-heavy chain disulfide bonds of the F(ab′)₂ fragment with mercaptoethanolamine (MEA). The exact reducing conditions were determined prior to conjugation in small-scale experiments. Size exclusion HPLC was performed and the profile is depicted for the Fab′ in FIG. 4. By this type of analysis the 5-9A7 Fab′ was >90% pure.

The Fab′ fragment of the 5-9A7 was separated from free MEA by G-25 column chromatography and mixed with 88.53 Fab-TNB at a 1:1 molar ratio overnight at room temperature. The HPLC profile depicted in FIG. 5 represents a profile of the conjugation mixture after 18 hours of incubation and before purification. This profile shows a mixture of bispecific antibody as well as unconjugated Fab′ molecules.

The bispecifc antibody was purified from contaminating Fab′ molecules by Superdex 200 size exclusion chromatography and the purified molecule was analyzed by HPLC. As shown in FIG. 6 the 88.53×5-9A7 bispecific antibody was purified to near homogeneity.

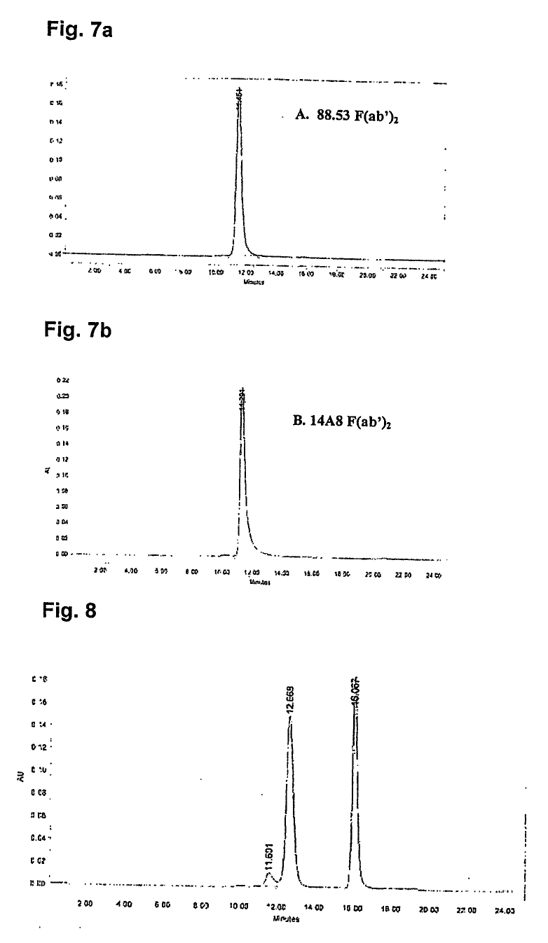

For control anti-CD64×anti-CD89 Bispecific Antibody were generated. F(ab′)₂ fragments of each of the HuMAbs, anti-CD64 (88.53), and anti-CD89 (14A8) were generated by pepsin digestion and purified to homogeneity by Superdex 200 gel filtration chromatography. Size exclusion HPLC was performed and profiles are depicted for each of the F(ab′)₂ in FIG. 7. By this type of analysis both of the F(ab′)₂ fragments were >95% pure.

A Fab′ fragment of the 88.53 was generated by mild reduction of the inter-heavy chain disulfide bonds of the F(ab′)₂ fragment with mercaptoethanolamine (MEA). The exact reducing conditions were determined prior to conjugation in small-scale experiments. Size exclusion HPLC was performed and the profile is depicted for the Fab′ in FIG. 8. By this type of analysis the 88.53 Fab′ was >90% pure.

The Fab′ fragment of the 88.53 was separated from free MEA by G-25 column chromatography. The Fab′ fragment was incubated with dinitrothiobenzoate (DTNB) 16a and 16b to generate a Fab-TNB conjugate.

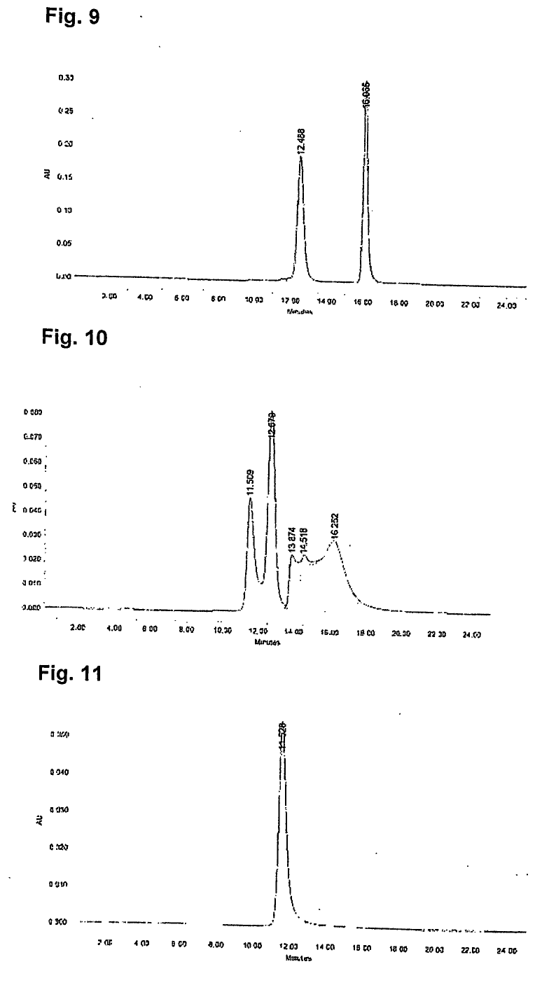

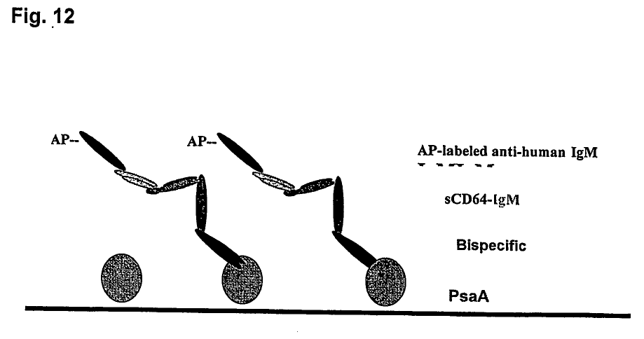

A Fab′ fragment of the 14A8 was generated by mild reduction of the inter-heavy chain disulfide bonds of the F(ab′)₂ fragment with mercaptoethanolamine (MEA). The exact reducing conditions were determined prior to conjugation in small-scale experiments. Size exclusion HPLC was performed and the profile is depicted for the Fab′ in FIG. 9. By this type of analysis the 14A8 Fab′ was >95% pure.

The Fab′ fragment of the 14A8 was separated from free MEA by G-25 column chromatography and mixed with 88.53 Fab-TNB at a 1:1 molar ratio overnight at room temperature. The HPLC profile depicted in FIG. 10 represents a profile of the conjugation mixture after 18 hours of incubation and before purification. This profile shows a mixture of bispecific antibody as well as unconjugated Fab′ molecules.

The bispecific antibody was purified from contaminating Fab′ molecules by Superdex 200 size exclusion chromatography and the purified molecule was analyzed by HPLC. As shown in FIG. 11 the 88.53×14A8 bispecific antibody was purified to near homogeneity.

Characterization of the Binding Specificity of the Anti-CD64×Anti-PsaA Bispecific Antibody—Bispecific ELISA
1. ELISA plates were coated recombinant PsaA, 50 μl/well, 5 μg/ml and incubated overnight at 4° C.
2. The plates were blocked with 5% BSA in PBS.
3. Titrations of the bispecific antibody were added to the plate. Controls included the anti-CD64×anti-CD89 bispecific (control bispecific) and the F(ab′)₂ fragments of the anti-CD64 Ab, 88.53 or of the anti-PsaA Ab, 5-9A7.
4. The plates were then incubated with a supernatant containing a fusion protein consisting of soluble CD64 linked to the Fc portion of human IgM.
5. The plates were finally incubated with an alkaline phosphatase labelled goat anti-human IgM antibody. Positive wells were detected with the alkaline phosphatase substrate.

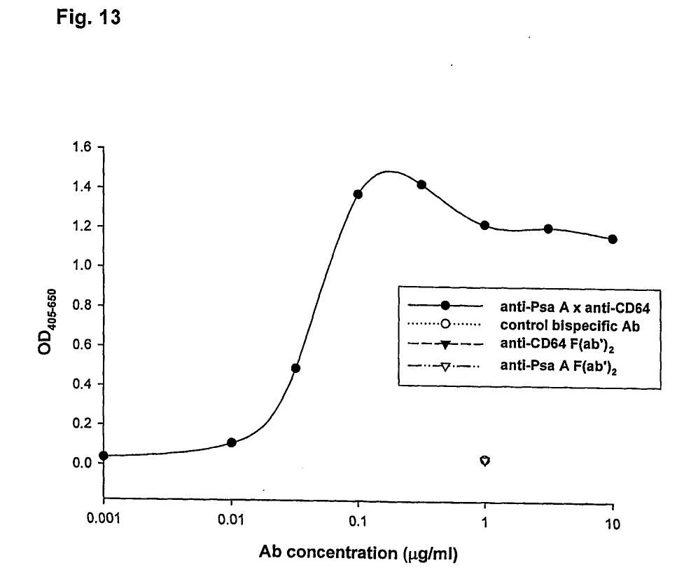

The anti-PsaA×anti-CD64 bispecific showed dose-dependent binding in this assay (see FIG. 13). The control bispecific was not detected since it does not bind the PsaA, the anti-CD64 F(ab′)₂ was not detected since it binds CD64 but not PsaA, and the anti-PsaA F(ab′)₂ was not detected since it binds PsaA but not the soluble CD64-IgM fusion protein.

Characterization of the Binding Specificity of the Anti-CD64×anti-PsaA Bispecific Antibody—Binding to CD64 on Human CD64-Transgenic Mice

Blood Was taken from CD64 transgenic mice or from non-transgenic littermates, and was incubated with the 88.53×5-9A7 bispecific antibody at a concentration of 30 μg/ml for 30 minutes at room temperature.

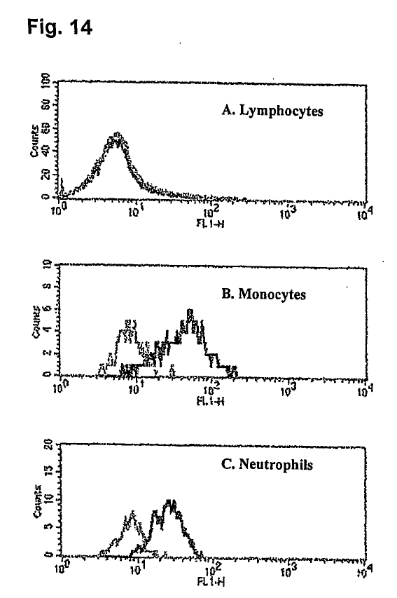

The blood was washed and then incubated with an FITC-labelled anti-human IgG antibody for 30 minutes at room temperature. The red blood cells were lysed and the remaining leukocytes were analyzed for staining by flow cytometry. Regions corresponding to the lymphocyte, monocyte, and neutrophil populations were gated and analyzed separately. The results of the binding assay are depicted in FIG. 14 (A-C). The black lines represent staining of cells from CD64 transgenic mice, the green lines cell from non-transgenic littermates.

Human CD64 is expressed on monocytes and, to a lesser extent, neutrophils of CD64 transgenic mice. As in humans, CD64 is not expressed by lymphocytes of the transgenic mice. The data in FIG. 14 show that the bispecific antibody binds to CD64 transgenic monocytes and neutrophils, but not to any cell populations derived from non-transgenic mice.

In summary, two bispecific antibodies, anti-CD64×anti-PsaA and anti-CD64×anti-CD89, were generated and purified to homogeneity. The 88.53×5-9A7 bispecific antibody was shown to bind simultaneously to CD64 and to PsaA. In addition this bispecific antibody binds to CD64 expressed by human CD64 transgenic mice.

### EXAMPLE 6

Sequencing of Antibodies

Antibodies shown in the affinity table In Example 3b were sequenced by conventional methods, and the variable regions of the antibodies are shown in the Figures.

9A7 (clone 5-9A7) FIGS. 16a and 16b

1G9 (clone 7-1G9.1) FIGS. 17a and 17b

15E5 (clone 1-15E5.2.1) FIGS. 18a and 18b

### EXAMPLE 7

Identification of Localisation of Epitopes

15 synthetic fragments of PsaA, each representing 25 amino acid residues of PsaA and overlapping with neighbouring fragments with 5 amino acid residues, were produced. Antibody binding to the fragments was tested in a standard ELISA assay. It was found that antibodies according to the invention bound to the N-terminal fragments corresponding to SEQ ID NOs 51, 52 and 53.

### REFERENCES

1. Alexander, J. E., R. A. Lock, C. A. M. Peeter, J. T. Poolman, P. W. Andrew, T. J. Mitchell, D. Hansman, and J. C. Paton. 1994. Immunizazation of mice with pneumolysin toxoid confers a significant degree of protection against at least nine serotypes of Streptococcus pneumoniae. Infect. Immun. 62:5683
2. AlonsoDeVelasco, E. Streptococcus pneumoniae: virulence factors, pathogenesis, and vaccines. Microbiol. Rev. December 1995. 59:591-603.
3. anonymous. 1985. Acute respiratory Infections in under fives: 15 million deaths a year. Lancet 2:699-701.
4. Au, C. C. and T. K. Eisenstein. 1981. Evaluation of the role of the pneumococcal Forssman antigen (F-polysaccharide) in the cross-serotype protection induced by pneumococal subcellular preparations. Infect. Immun. 31:169-173.
5. Barry, A. M., R. A. Lock, D. Hansman, and J. C. Paton. 1989. Contribution of autolysin to virulence of Streptococcus pneumoniae. Infect. Immun. 57:2324-2330.
6. Briles D E, et al. Mouse antibody to phosphocholine can protect mice from infection with mouse-virulent human isolates of Streptococcus pneumoniae. Infect. Immun. May 1992. 60:1957-1962.
7. Camara, M., G. J. Boulnois, P. W. Andrew, and T. J. Mitchell. 1994. A neuraminidase from Streptococcus pneumoniae has the features of a surface protein. Infect. Immun. 62:3688-3695.
8. Crain M J, et al. Pneumococcal surface protein A (PspA) is serologically highly variable and is expressed by all clinically important capsular serotypes of Streptococcus pneumoniae. Infect. Immun. October 1990. 58:3293-3299.
9. Crook, J., J. A. Tharpe, S. E. Johnson, D. Williams, A. R. Stinson, R. Facklam, E. W. Ades, G. M. Carlone, and J. S. Sampson. 1998. Immunoreactivity of five monoclonal antibodies against the 37-kilodalton common cell wall protein (PsaA) of Streptococcus pneumoniae. Clin. Diagn. Lab. Immunol. 5:205-210.
10. De B K, et al. Baculovirus expression, purification and evaluation of recombinant pneumococcal surface adhesin A of streptococcus pneumoniae. Pathobiology. May-June 1999. 67:115-122.
11. de los Toyos J R, et al. Functional analysis of pneumolysin by use of monoclonal antibodies. Infect. Immun. February 1996. 64:480-484.
12. Gillespie, S. H., P. H. M. McWhinney, S. Patel, J. G. Raynes, K. P. W. J. McAdam, R. A. Whiley, and J. M. Hardie. 1993. Species of alpha-hemolytic streptococci possessing a C-polysaccharide phosphorylcholine-containing antigen. Infect. Immun. 61:3076-3077.
13. Kuo, J., M. Douglas, H. K. Ree, and A. A. Lindberg. 1995. Characterization of a recombinant pneumolysin and its use as a protein carrier for pneumococcal type 18C conjugate vaccines. Infect. Immun. 63:2706-2713.
14. Lee C J, et al. Immunologic epitope, gene, and Immunity involved in pneumococcal glycoconjugate. Crit. Rev. Microbiol. 1997. 23:121-142.
15. Lock, R. A., J. C. Paton, and D. Hansman. 1988. Comparative efficacy of pneumococcal neuraminidase and pneumolysin as immunogens protective against Streptococcus pneumonlae. Microb. Pathog. 5:461-467.
16. Lomholdt, H. 1995. Evidence of recombination and an antigenically diverse immunoglobulin A1 protease among strains of Streptococcus pneumoniae. Infect. Immun. 63:4238-4243.
17. McDaniel L S, et al. Monoclonal antibodies against protease-sensitive pneumococcal antigens can protect mice from fatal infection with Streptococcus pneumoniae. J. Exp. Med. August 1, 1984. 160:386-397.
18. McDaniel L S, et al. PspA, a surface protein of Streptococcus pneumoniae, is capable of eliciting protection against pneumococci of more than one capsular type. Infect. Immun. January 1991. 59:222-228.
19. Mitchell, T. J., F. Mendez, J. C. Paton, P. W. Andrew, and G. J. Boulnols. 1990. Comparison of pneumolysin genes and proteins from Streptococcus pneumoniae type 1 and 2. Nuclei. Acids. Res. 18:4010
20. Nielsen S V, et al. Antibodies against pneumococcal C-polysaccharide are not protective. Microb. Pathog. April 1993. 14:299-305.
21. Paton, J. C., R. A. Lock, and D. Hansman. 1983. Effect of immunization with pneumolysin on survival time of mice challenged with Streptococcus pneumoniae. Infect. Immun. 40:548
22. Paton, J. C., R. A. Lock, C. J. Lee, J. P. L I, A. M. Barry, T. J. Mitchell, P. W. Andrew, D. Hansman, and G. J. Boulnois. 1991. Purification and immunogenicity of genetically obtained pneumolysin toxoids and their conjugation to Streptococcus pneumoniae type 19F polysaccharide. Infect. Immun. 59:2297-2304.
23. Russell, H., J. A. Tharpe, D. E. Wells, E. H. White, and J. E. Johnson. 1990. Monoclonal antibody recognizing a species-specific protein from Streptococcus pneumoniae. J. Clin. Microbiol. 28:2191-2195.
24. Sampson, J. S., Z. Furlow, A. M. Whitney, D. Williams, R. Facklam, and G. M. Carlone. 1997. Limited diversity of Streptococcus pneumoniae psaA among pneumococcal vaccine serotypes. Infect. Immun. 65:1967-1971.
25. Sampson, J. S., S. P. O'Connor, A. R. Stinson, J. A. Tharpe, and H. Russell. 1994. Cloning and nucleotide sequence of psaA, the Streptococcus pneumoniae gene encoding a 37-kilodalton protein homologous to previously reported Steptococcus sp. adhesin. Infect. Immun. 62:319-324.
26. Sorensen, U. B. Pneumococcal polysaccharide antigens: capsules and C-polysaccharide. An immunochemical study. Dan. Med. Bull. February 1995. 42:47-53.
27. Swiatlo, E., M. J. Crain, L. S. McDaniel, A. Brooks-Walter, T. J. Coffey, B. G. Spratt, D. A. Morrison, and D. E. Briles. 1996. DNA polymorphisms and variants penicillin-binding proteins as evidence that relatively penicillin-resistant pneumococci in Western Canada are clonally related. J. Infect. Dis. 174:884-888.
28. Szu S C, et al. Rabbit antibodies to the cell wall polysaccharide of Streptococcus pneumoniae fall to protect mice from lethal challenge with encapsulated pneumococci. Infect. Immun. November 1986. 54:448455.
29. Sørensen, U. B. S. and J. Henrichsen. 1987. Cross-reactions between pneumococci and other streptococci due to C-polysaccharide and F-antigen. J. Clin. Microbiol. 25:1854-1859.
30. Tai, S. S., T. R. Wang, and C. J. Lee. 1997. Characterization of hemin binding activity of Streptococcus pneumoniae. Infect. Immun. 65:1083
31. Talkington D F, et al. A 43-kilodalton pneumococcal surface protein, PspA: isolation, protective abilities, and structural analysis of the amino-terminal sequence. Infect. Immun. April 1991. 59:1285-1289.
32. Talkington, D. F., B. G. Brown, J. A. Tharpe, A. Koenig, and H. Russell. 1996. Protection of mice against fatal pneumococcal challenge by immunization with pneumococcal surface adhesin A (PsaA). Microb. Pathog. 21:17-22.
33. Tharpe, J. A. and H. Russell. 1996. Purification and seroreactivity of pneumococcal surface adhesin A (PsaA). Clin. Diagn. Lab. Immunol. 3:227-229.
34. Simell, B., Korkella, M., Pursiainen, H., Kilpl, T. M., and Käyhty, H. 2001. Pneumococcal carriage and otitis media induce salivary antibodies to pneumococcal surface adhesin A, pneumolysin, and pneumococcal surface protein A In children. J. Infect. Dis. 183:887-896.
35. Rapola, S., Jännti, V., Haikala, R., Carlone, G. M., Sampson, J. S., Briles, D. E., Paton, J. C., Takala, A. K., Kilpi, T. M., and Käyhty, H. 2000. Natural development of antibodies to pneumococcal surface protein A, pneumococal surface adhesin A, and pneumolysin in relation to pneumococcal carriage and acute otitis media. J. Infect. Dis. 182:1146-1152.

1. An isolated binding member comprising at least one binding domain capable of specifically binding Streptococcus pneumoniae surface adhesin A (PsaA) protein, said binding domain having a dissociation constant K_{d} for PsaA which is less than 1×10⁻⁶M.

2. The isolated binding member according to claim 1, wherein the isolated binding member is a pure isolated binding member.

3. The isolated binding member according to claim 1, wherein the binding member is selected from antibodies or immunologically active fragments of antibodies or single chain of antibodies.

4. The isolated binding member according to claim 3, wherein the antibodies are selected from monoclonal antibodies, polyclonal antibodies or mixtures of monoclonal antibodies.

5. The isolated binding member according to claim 1, wherein the binding member is monospecific towards the PsaA protein.

6. The isolated binding member according to claim 1, wherein the binding member is bispecific having at least one portion specific towards the PsaA protein.

7. The isolated binding member according to claim 1, wherein the binding member is multispecific having at least one portion towards the PsaA protein.

8. The isolated binding member according to claim 1, wherein the binding domain is carried by a human antibody framework.

9. The Isolated binding member according to claim 1, wherein the binding domain is carried by a humanised antibody framework.

10. The isolated binding member according to any of the preceding claims, wherein said binding domain recognizes an epitope in the N-terminal part of PsaA.

11. The isolated binding member according to any of the preceding claims, wherein said binding domain recognizes an epitope in the N-terminal 100 amino acid residues of PsaA.

12. The isolated binding member according to any of the preceding claims, wherein the binding domain comprises an amino acid sequence selected from SEQ ID NO 2, from SEQ ID NO 4, from SEQ ID NO 6, and from SEQ ID NO 8 or a homologue thereof.

13. The isolated binding member according to claim 12, wherein the binding domain comprises at least two amino acid sequences selected from SEQ ID NO 2, from SEQ ID NO 4, from SEQ ID NO 6, and from SEQ ID NO 8 or a homologue thereof.

14. The isolated binding member according to claim 12, wherein the binding domain comprises at least SEQ ID NO 4, and SEQ ID NO 6, or a homologue thereof.

15. The isolated binding member according to claim 12, wherein the binding domain comprises SEQ ID NO 2, SEQ ID NO 4, and SEQ ID NO 6, or a homologue thereof.

16. The isolated binding member according to claim 12, wherein the binding domain comprises SEQ ID NO 8, or a homologue thereof.

17. The isolated binding member according to any of the preceding claims, wherein the binding member is capable of binding PsaA from two or more different Pneumococcus serotypes.

18. The isolated binding member according to any one of claims 12-17, wherein the homologue is at least 60% homologous to one or more of the sequences selected from SEQ ID NO 2, from SEQ ID NO 4, from SEQ ID NO 6, and from SEQ ID NO 8, such as at least 65% homologous such as at least 70% homologous, such as at least 75% homologous, 'such as at least 80% homologous, such as at least 85% homologous, such as at least 90% homologous, such as at least 95% homologous, such as at least 98% homologous.

19. The isolated binding member according to any of the preceding claims, wherein said binding member is capable of binding to an epitope on PsaA, said epitope being recognized by the binding member as defined in any one of claims 12-16.

20. The isolated binding member according to claim 1, wherein the dissociation constant is less than 5×10⁻⁹ M, such as less than 1×10⁻⁹ M.

21. The isolated binding member according to any of the preceding claims, wherein the binding domain is located in a V_{L} domain.

22. The isolated binding member according to any of the preceding claims, wherein the binding domain is located in a V_{H} domain.

23. The isolated binding member according to any one of claims 12-15, wherein the binding domain is arranged as a complementarity-determining region (CDR) in the binding member.

24. The isolated binding member according to claim 2, wherein the fragment of antibodies are selected from Fab, Fab′, F(ab)₂ and Fv.

25. The binding member according to any of the preceding claims, comprising at least a first binding domain and a second binding domain, said first binding domain being capable of specifically binding Streptococcus pneumoniae surface adhesin A (PsaA) protein, and said second binding domain is different from said first binding domain.

26. The isolated binding member according to claim 25, wherein the second binding domain is capable of specifically binding a mammalian protein, such as a human protein, such as a protein selected from CD64 or CD89.

27. The isolated binding member according to claim 25, wherein the second binding domain is capable of specifically binding a mammalian cell, such as a human cell, such as a cell selected from a leucocyte, macrophages, lymphocytes, neutrophilic cells, basophilic cells, and eosinophilic cells.

28. The isolated binding member according to claim 26, wherein the second binding domain is capable of specifically binding a Pneumococcus protein.

29. The isolated binding member according to claim 28, wherein second binding domain is capable of specifically binding a PsaA epitope different from the first binding domain.

30. The isolated binding member according to claim 25, wherein the binding member comprises two binding domains.

31. The isolated binding member according to claim 30, wherein the two binding members are linked through a spacer region.

32. An isolated nucleic acid molecule encoding at least a part of the binding member as defined in any one of claims 1-31.

33. A vector comprising the nucleic acid molecule as defined in claim 32.

34. The vector according to claim 33, comprising a nucleotide sequence which regulates the expression of the antibody encoded by the nucleic acid molecule.

35. A host cell comprising the nucleic acid molecule as defined in claim 32.

36. A cell line engineered to express the binding member as defined in any of claims 1-31.

37. A method of detecting of diagnosing a disease or disorder associated with Pneumococcus in an individual comprising
providing a biological sample from said individual,
adding at least one binding member as defined in any of claims 1-31 to said biological sample,
detecting binding members bound to said biological sample,.thereby detecting or diagnosing the disease or disorder.

38. A kit comprising at least one binding member as defined in any of claims 1-31, said antibody being labelled.

39. A pharmaceutical composition comprising at least one binding member as defined in any of claims 1-31.

40. The pharmaceutical composition according to claim 39, comprising at least two different binding members.

41. Use of a binding member as defined in any of claims 1-31 for the production of a pharmaceutical composition.

42. Use of a binding member as defined in any of claims 1-31 for the production of a pharmaceutical composition for the treatment of Pneumococcus infection.


SEQUENCE LISTING (56 sequences)

SEQ ID NO: 1 - DNA, 33 bp, Homo sapiens
  CDS (1)..(33): Sequence from human antibody generated in mouse.
cgggcgagtcagggtattagcagctggttagcc

SEQ ID NO: 2 - protein, 11 aa, Homo sapiens
RASQGISSWLA

SEQ ID NO: 3 - DNA, 21 bp, Homo sapiens
  CDS (1)..(21): Sequence from human antibody generated in mouse.
gttgcatccagtttgcaaagt

SEQ ID NO: 4 - protein, 7 aa, Homo sapiens
VASSLQS

SEQ ID NO: 5 - DNA, 27 bp, Homo sapiens
  CDS (1)..(27): Sequence from human antibody generated in mouse.
caacagtataatagctatcctccgacg

SEQ ID NO: 6 - protein, 9 aa, Homo sapiens
QQYNSYPPT

SEQ ID NO: 7 - DNA, 321 bp, Homo sapiens
  CDS (1)..(321): Sequence from human antibody generated in mouse.  V-segment 4-34 and J-segment JK1
  CDR1 (70)..(120)
  CDR2 (148)..(168)
  CDR3 (265)..(291)
gacatccagatgacccagtctccatcctcactgtctgcatctgtaggagacagagtcaccatcacttgtcgggcgagtcagggtattagcagctggttagcctggtatcagcagaaaccagagaaagcccctgagtccctgatctatgttgcatccagtttgcaaagtggggtcccatcaaggttcagcggcagtggatctgggacagatttcactctcaccatcagcagcctgcagcctgaagattttgcaacttattactgccaacagtataatagctatcctccgacgttcggccaagggaccaaggtggaaatcaaa

SEQ ID NO: 8 - protein, 107 aa, Homo sapiens
DIQMTQSPSSLSASVGDRVTITCRASQGISSWLAWYQQKPEKAPESLIYVASSLQSGVPSRFSGSGSGTDFTLTISSLQPEDFATYYCQQYNSYPPTFGQGTKVEIK

SEQ ID NO: 9 - DNA, 15 bp, Homo sapiens
  CDS (1)..(15): Sequence from human antibody generated in mouse.
ggtttctcctggagc

SEQ ID NO: 10 - protein, 5 aa, Homo sapiens
GFSWS

SEQ ID NO: 11 - DNA, 51 bp, Homo sapiens
  CDS (1)..(51): Sequence from human antibody generated in mouse.
gaaatcgattatagaggaagcaccaactacaacccgtccctcaagagtcga

SEQ ID NO: 12 - protein, 17 aa, Homo sapiens
EIDYRGSTNYNPSLKSR

SEQ ID NO: 13 - DNA, 21 bp, Homo sapiens
  CDS (1)..(21): Sequence from human antibody generated in mouse.
ggggggccccgctttgactac

SEQ ID NO: 14 - protein, 7 aa, Homo sapiens
GGPRFDY

SEQ ID NO: 15 - DNA, 345 bp, Homo sapiens
  CDS (1)..(345): Sequence from human antibody generated in mouse. V-segment 4-34, D-segment unknown, J-segment JH4b
  CDR1 (91)..(102)
  CDR2 (148)..(199)
  CDR3 (191)..(312)
caggtgcgactacagcagtggggcgcaggactgttgaagccttcggagaccctgtccctcacctgcgctgtctttggtgggtccttcagtggtttctcctggagctggatccgccagaccccagggaaggggctggagtggatcggggaaatcgattatagaggaagcaccaactacaacccgtccctcaagagtcgagtcaccatattaagagacacgtccaggagccagttctccctgaagttgagctccgtgaccgccgcggactcggctgtgttttattgtgcgagaggggggccccgctttgactactggggccagggaaccctggtcaccgtctcctca

SEQ ID NO: 16 - protein, 115 aa, Homo sapiens
QVRLQQWGAGLLKPSETLSLTCAVFGGSFSGFSWSWIRQTPGKGLEWIGEIDYRGSTNYNPSLKSRVTILRDTSRSQFSLKLSSVTAADSAVFYCARGGPRFDYWGQGTLVTVSS

SEQ ID NO: 17 - DNA, 33 bp, Artificial
  synthetic
  CDS (1)..(33)
  misc_feature (33)..(33): unknown nucleotide
agggccagtcagagtgttagcagctacttagcn

SEQ ID NO: 18 - protein, 11 aa, Artificial
  Synthetic Construct
RASQSVSSYLA

SEQ ID NO: 19 - DNA, 21 bp, Homo sapiens
  CDS (1)..(21): Sequence from human antibody generated in mouse.
gatgcatccaacagggccact

SEQ ID NO: 20 - protein, 7 aa, Homo sapiens
DASNRAT

SEQ ID NO: 21 - DNA, 27 bp, Homo sapiens
  CDS (1)..(27): Sequence from human antibody generated in mouse.
cagcagcgtagcaactggcctctcact

SEQ ID NO: 22 - protein, 9 aa, Homo sapiens
QQRSNWPLT

SEQ ID NO: 23 - DNA, 321 bp, Homo sapiens
  CDS (1)..(318): Sequence from human antibody generated in mouse.  V-segment L6 and J-segment JK4
  CDR1 (70)..(102)
  misc_feature (102)..(102): unknown nucleotide
  CDR2 (148)..(168)
  CDR3 (265)..(291)
gaaattgtgttgacacagtctccagccaccctgtctttgtctccaggggaaagagccaccctctcctgcagggccagtcagagtgttagcagctacttagcntggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctctcactttcggcggagggaccaaggtggagatcaaa

SEQ ID NO: 24 - protein, 106 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEI

SEQ ID NO: 25 - DNA, 15 bp, Artificial
  synthetic
  CDS (1)..(15)
atctttgggatgagc

SEQ ID NO: 26 - protein, 5 aa, Artificial
  Synthetic Construct
IFGMS

SEQ ID NO: 27 - DNA, 51 bp, Homo sapiens
  CDS (1)..(51): Sequence from human antibody generated in mouse.
aacataaagcaagatggaagtgagaaatactatgtggactctgtgaagggc

SEQ ID NO: 28 - protein, 17 aa, Homo sapiens
NIKQDGSEKYYVDSVKG

SEQ ID NO: 29 - DNA, 57 bp, Homo sapiens
  CDS (1)..(57): Sequence from human antibody generated in mouse.
gatcggttttactatggttcggggagttattattactactacaacggtatggacgtc

SEQ ID NO: 30 - protein, 19 aa, Homo sapiens
DRFYYGSGSYYYYYNGMDV

SEQ ID NO: 31 - DNA, 384 bp, Homo sapiens
  CDS (1)..(384): Sequence from human antibody generated in mouse. V-segment3-7, d-segment 3-10 and J-segment JH6b
  CDR1 (91)..(102)
  CDR2 (148)..(198)
  CDR3 (295)..(351)
gaggtgcaactagtggagtctgggggaggcttggtccagcctggggggtccctgagactctcctgtgcagcctctggattcacctttaatatctttgggatgagctgggtccgccaggctccagggaaagggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagggatcggttttactatggttcggggagttattattactactacaacggtatggacgtctggggccaagggaccacggtcaccgtctcctca

SEQ ID NO: 32 - protein, 128 aa, Homo sapiens
EVQLVESGGGLVQPGGSLRLSCAASGFTFNIFGMSWVRQAPGKGLEWVANIKQDGSEKYYVDSVKGRFTISRDNAKNSLYLQMNSLRAEDTAVYYCARDRFYYGSGSYYYYYNGMDVWGQGTTVTVSS

SEQ ID NO: 33 - DNA, 33 bp, Homo sapiens
  CDS (1)..(33): Sequence from human antibody generated in mouse.
agggccagtcagagtgttagcagctacttagcc

SEQ ID NO: 34 - protein, 11 aa, Homo sapiens
RASQSVSSYLA

SEQ ID NO: 35 - DNA, 21 bp, Homo sapiens
  CDS (1)..(21): Sequence from human antibody generated in mouse.
gatgcatccaacagggccact

SEQ ID NO: 36 - protein, 7 aa, Homo sapiens
DASNRAT

SEQ ID NO: 37 - DNA, 30 bp, Homo sapiens
  CDS (1)..(30): Sequence from human antibody generated in mouse.
cagcagcgtagcaactggcctccattcact

SEQ ID NO: 38 - protein, 10 aa, Homo sapiens
QQRSNWPPFT

SEQ ID NO: 39 - DNA, 324 bp, Homo sapiens
  CDS (1)..(324): Sequence from human antibody generated in mouse. V-segment L6 and J-segment JK3
  CDR1 (70)..(102)
  CDR2 (148)..(168)
  CDR3 (265)..(294)
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccattcactttcggccctgggaccaaagtggatatcaaa

SEQ ID NO: 40 - protein, 108 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGPGTKVDIK

SEQ ID NO: 41 - DNA, 15 bp, Homo sapiens
  CDS (1)..(15): Sequence from human antibody generated in mouse.
agcttttggatgagc

SEQ ID NO: 42 - protein, 5 aa, Homo sapiens
SFWMS

SEQ ID NO: 43 - DNA, 30 bp, Homo sapiens
  CDS (1)..(30): Sequence from human antibody generated in mouse.
aacataaagcaagatggaagtgagaaattc

SEQ ID NO: 44 - protein, 10 aa, Homo sapiens
NIKQDGSEKF

SEQ ID NO: 45 - DNA, 54 bp, Homo sapiens
  CDS (1)..(54): Sequence from human antibody generated in mouse.
gatcgtattacaatggttcggccctattactacttctacaacggtctggacgtc

SEQ ID NO: 46 - protein, 18 aa, Homo sapiens
DRITMVRPYYYFYNGLDV

SEQ ID NO: 47 - DNA, 381 bp, Homo sapiens
  CDS (1)..(381): Sequence from human antibody generated in mouse. V-segment 3-7, D-segment 3-10 and J-segment JH6b
  CDR1 (91)..(102)
  CDR2 (148)..(177)
  CDR3 (295)..(348)
gaggtacagctggtggagtctgggggaggcttggtccagccgggggggtccctgagactctcctgtgcagcttctggattcacctttagtagcttttggatgagctgggtccgccaggctccagggaaggggctggagtgggtggccaacataaagcaagatggaagtgagaaattctatgtggactctgtgaagggccgattcaccatctccagagacaacgccaagaactcactgtatctgcaaatgaacagcctgagagccgaggacacggctgtgtattactgtgcgagggatcgtattacaatggttcggccctattactacttctacaacggtctggacgtctggggccaagggaccacggtcaccgtctcctca

SEQ ID NO: 48 - protein, 127 aa, Homo sapiens
EVQLVESGGGLVQPGGSLRLSCAASGFTFSSFWMSWVRQAPGKGLEWVANIKQDGSEKFYVDSVKGRFTISRDNAKNSLYLQMNSLRAEDTAVYYCARDRITMVRPYYYFYNGLDVWGQGTTVTVSS

SEQ ID NO: 49 - DNA, 930 bp, Streptococcus pneumoniae
  CDS (1)..(930): Sequence of Streptococcus pneumoniae surface adhesin A (PsaA)- A Variant
atgaaaaaattaggtacattactcgttctctttctttctgcaatcattcttgtagcatgtgctagcggaaaaaaagatacaacttctggtcaaaaactaaaagttgttgctacaaactcaatcatcgctgatattactaaaaatattgctggtgacaaaattgaccttcatagtatcgttccgattgggcaagacccacacgaatacgaaccacttcctgaagacgttaagaaaacttctgaggctgatttgattttctataacggtatcaaccttgaaacaggtggcaatgcttggtttacaaaattggtagaaaatgccaagaaaactgaaaacaaagactacttcgcagtcagcgacggcgttgatgttatctaccttgaaggtcaaaatgaaaaaggaaaagaagacccacacgcttggcttaaccttgaaaacggtattatttttgctaaaaatatcgccaaacaattgagcgccaaagaccctaacaataaagaattctatgaaaaaaatctcaaagaatatactgataagttagacaaacttgataaagaaagtaaggataaatttaataagatccctgctgaaaagaaactcattgtaaccagcgaaggagcattcaaatacttctctaaagcctatggtgttccaagtgcctacatctgggaaatcaatactgaagaagaaggaactcctgaacaaatcaagaccttggttgaaaaacttcgccaaacaaaagttccatcactctttgtagaatcaagtgtggatgaccgtccaatgaaaactgtttctcaagacacaaacatcccaatctacgcacaaatctttactgactctatcgcagaacaaggtaaagaaggcgacagctactacagcatgatgaaatacaaccttgacaagattgctgaaggattggcaaaataa

SEQ ID NO: 50 - protein, 309 aa, Streptococcus pneumoniae
MKKLGTLLVLFLSAIILVACASGKKDTTSGQKLKVVATNSIIADITKNIAGDKIDLHSIVPIGQDPHEYEPLPEDVKKTSEADLIFYNGINLETGGNAWFTKLVENAKKTENKDYFAVSDGVDVIYLEGQNEKGKEDPHAWLNLENGIIFAKNIAKQLSAKDPNNKEFYEKNLKEYTDKLDKLDKESKDKFNKIPAEKKLIVTSEGAFKYFSKAYGVPSAYIWEINTEEEGTPEQIKTLVEKLRQTKVPSLFVESSVDDRPMKTVSQDTNIPIYAQIFTDSIAEQGKEGDSYYSMMKYNLDKIAEGLAK

SEQ ID NO: 51 - protein, 25 aa, Streptococcus pneumoniae
MKKLGTLLVLFLSAIILVACASGKK

SEQ ID NO: 52 - protein, 25 aa, Streptococcus pneumoniae
ASGKKDTTSGQKLKVVATNSIIADI

SEQ ID NO: 53 - protein, 25 aa, Streptococcus pneumoniae
IIADITKNIAGDKIDLHSIVPIGQD

SEQ ID NO: 54 - protein, 65 aa, Streptococcus pneumoniae
MKKLGTLLVLFLSAIILVACASGKKDTTSGQKLKVVATNSIIADITKNIAGDKIDLHSIVPIGQD

SEQ ID NO: 55 - DNA, 960 bp, Streptococcus pneumoniae
  CDS (1)..(930): equence of Streptococcus pneumoniae surface adhesin A (PsaA)
atgaaaaaattaggtacattactcgttctctttctttctgcaatcattcttgtagcatgtgctagcggaaaaaaagatacaacttctggtcaaaaactaaaagttgttgctacaaactcaatcatcgctgatattactaaaaatattgctggtgacaaaattgaccttcatagtatcgttccgattgggcaagacccacacgaatacgaaccacttcctgaagacgttaagaaaacttctgaggctgatttgattttctataacggtatcaaccttgaaacaggtggcaatgcttggtttacaaaattagtagaaaatgccaagaaaactgaaaacaaagactacttcgcagtcagcgacggcgttgatgttatctaccttgaaggtcaaaatgaaaaaggaaaagaagacccacacgcttggcttaaccttgaaaacggtattatttttgctaaaaatatcgccaaacaattgagcgccaaagaccctaacaataaagaattctatgaaaaaaatctcaaagaatatactgataagttagacaaacttgataaagaaagtaaggataaatttaataagatccctgctgaaaagaaactcattgtaaccagcgaaggagcattcaaatacttctctaaagcctatggtgttccaagtgcctacatctgggaaatcaatactgaagaagaaggaactcctgaacaaatcaagaccttggttgaaaaacttcgccaaacaaaagttccatcactctttgtagaatcaagtgtggatgaccgtccaatgaaaactgtttctcaagacacaaacatcccaatctacgcacaaatctttactgactctatcgcagaacaaggtaaagaaggcgacagctactacagcatgatgaaatacaaccttgacaagattgctggaggattggcaaaataagacaagattgctgaaggattggcaaaataa

SEQ ID NO: 56 - protein, 309 aa, Streptococcus pneumoniae
MKKLGTLLVLFLSAIILVACASGKKDTTSGQKLKVVATNSIIADITKNIAGDKIDLHSIVPIGQDPHEYEPLPEDVKKTSEADLIFYNGINLETGGNAWFTKLVENAKKTENKDYFAVSDGVDVIYLEGQNEKGKEDPHAWLNLENGIIFAKNIAKQLSAKDPNNKEFYEKNLKEYTDKLDKLDKESKDKFNKIPAEKKLIVTSEGAFKYFSKAYGVPSAYIWEINTEEEGTPEQIKTLVEKLRQTKVPSLFVESSVDDRPMKTVSQDTNIPIYAQIFTDSIAEQGKEGDSYYSMMKYNLDKIAGGLAK